(19) United States
(12) Patent Application Publication
(10) Pub. No.: US 2019/0125702 A1
(43) Pub. Date: May 2, 2019

(54) Methods and Compositions for the Treatment of Demyelinating Disorders

(71) Applicant: Vitality Biopharma, Inc., Los Angeles, CA (US)

(72) Inventors: Tassie Collins, San Mateo, CA (US); Karen Lariosa-Willingham, Mountain View, CA (US); Jason Dugas, San Carlos, CA (US); Jay S. Tung, Belmont, CA (US); Dmitri Leonoudakis, Pleasonton, CA (US); Elen Rosler, Mountain View, CA (US)

(73) Assignee: Vitality Biopharma, Inc., Los Angeles, CA (US)

(21) Appl. No.: 16/084,086

(22) PCT Filed: Mar. 13, 2017
(86) PCT No.: PCT/US17/22034

§ 371 Date: Sep. 11, 2018

Related U.S. Application Data
(60) Provisional application No. 62/308,814, filed on Mar. 15, 2016.

(51) Int. Cl. A61K 31/165; A61K 45/06; A61P 25/00; A61P 37/06

(52) U.S. Cl. CPC A61K 31/165; A61P 37/06; A61P 25/00; A61K 45/06

(57) ABSTRACT

The present invention provides methods and compositions for repairing and/or maintaining the myelin sheath of neuronal axons in a subject. In particular, the present invention provides compositions comprising one or more TRPV1 agonists exhibiting promyelinating activity for treatment of demyelinating disorders.

### REFERENCE TO RELATED APPLICATIONS

The present application claims priority to Provisional U.S. Application No. 62/308,814, filed March 15, 2016, entitled “METHODS AND COMPOSITIONS FOR THE TREATMENT OF DEMYELINATING DISORDERS,” and is incorporated herein in its entirety.

### FIELD OF THE INVENTION

The present invention relates to the treatment of demyelinating disorders. In particular, the present invention relates to the treatment of demyelinating disorders such as multiple sclerosis with therapeutic(s) which promote myelination alone or in combination with other therapeutics.

### BACKGROUND

Myelin is an electrically insulating material which encases the axons of neurons forming a layer known as the myelin sheath. The primary purpose of myelin is to increase the speed at which nerve impulses propagate down the neural axon. By increasing the electrical resistance across the cell membrane, myelin helps prevent the electrical current from leaving the axon. Neural demyelination is a condition characterized by a reduction of the myelin sheath in the nervous system, and is the basis for many neurodegenerative diseases or injuries, including but not limited to multiple sclerosis.

Multiple sclerosis (MS) is the most common disabling neurological disease of young adults; once established, it persists for the remainder of a person's life [1]. The initial triggering events which lead to MS remain unknown and there is no cure. In MS, central nervous system (CNS) lesions form as a result of immune-mediated destruction of myelin sheaths, resulting in loss of function and, ultimately, progressive neurodegeneration and permanent neurological decline. Current MS therapeutics mainly target the autoimmune response that damages myelin sheaths. Although effective in reducing relapses in early disease, or in some cases to provide symptomatic relief of pain or muscle spasticity, none of these treatments prevent long-term disease progression altogether, and very few have shown signs that they may be effective in treating progressive forms of the disease.

A major unmet medical need in the treatment of MS is the availability of therapeutics that directly protect myelin or promote new myelin formation to maintain nerve function, to prevent neurodegeneration, and to restore lost function in patients.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

The present invention provides methods and compositions for the treatment of demyelinating disorders. In one aspect of the invention, there is provided a method of repairing and/or maintaining the myelin sheath of neuronal axons in a subject, the method comprising administering an effective amount of one or more TRPV1 agonists exhibiting promyelinating activity.

In another aspect of the invention, there is provided a method of promoting myelination of an axon of a nerve cell, the method comprising contacting the nerve cell with an effective amount of one or more TRPV1 agonists exhibiting promyelinating activity.

In another aspect of the invention, there is provided a method of treating a demyelinating disorder in a subject, the method comprising administering an effective amount of one or more TRPV1 agonists exhibiting promyelinating activity.

In another aspect of the invention, there is provided a method of neuroprotection comprising administering to a subject an effective amount of one or more TRPV1 agonists exhibiting promyelinating activity alone or in combination with other therapeutics.

In specific embodiments, the one or more TRPV1 agonists exhibiting promyelinating activity are selected from the group consisting of zu-capsaicin, capsaicin, cannabinoids, such as cannabidivarin and cannabidiol, anadamide, vanilloids and combinations thereof.

In specific embodiments, the methods further comprise administration of one or more other therapeutics including but not limited to the one or more other therapeutics are selected from the group consisting of anti-inflammatory agents, immune modulators, other agents having promyelinating activity.

Other embodiments and advantages of the invention are set forth in part in the description, which follows, and in part, may be obvious from this description, or may be learned from the practice of the invention.

### DEFINITIONS AND ABBREVIATIONS:

As used herein, the term “demyelinating disorder” encompasses any neurological disorder or disease associated with the destruction or removal of myelin or myelin deficiency.

As used herein, the terms “treat”, “treatment”, and the like mean to relieve or alleviate at least one symptom associated with such condition, or to slow or reverse the progression of such condition. The term “treat” also denotes to arrest, delay the onset (i.e., the period prior to clinical manifestation of a disease) of a disease. For example, in relation to neurological disorders characterized by myelin loss or myelin deficiency, the term “treat” may mean to delay manifestation, arrest the progression, relieve or alleviate at least one symptom of the neurological disorder such as, but not limited to, impaired vision or cognitive function, numbness, weakness in extremities, tremors or spasticity, heat intolerance, speech impairment, incontinence, dizziness, impaired proprioception (e.g., balance, sense of limb position) or coordination, pain, memory, depression, and gait disorders.

As used herein, the term “promyelination activity” refers to the generation of myelin sheaths and/or promote remyelination. Promyelination activity can be monitored by methods known in the art which include direct determination of the state of myelin in a subject, e.g., one can measure white matter mass using magnetic resonance imaging (MRI), measure the thickness of myelin fibers using a magnetic resonance spectroscopy (MRS) brain scan, or any other direct measures known in the art (e.g., Positron-Emission Tomography (PET), Diffusion-Weighted Imaging (DW-I, or DW-MRI), Diffusion Tensor Imaging, Myelography, Magnetization Transfer, etc.). In vitro myelination assays may also be used to identify therapeutics having promyelination activity.

As used herein, the term “effective amount” is an amount of a therapeutic that is sufficient to reduce the occurrence of demyelination or increase the occurrence of remyelination in a mammalian recipient by at least 10% (e.g., 10%, 15%, 20%, 25%, 30%, 40%, 50%, 60%, 70%, 75%, 80%, 85%, 90%, 95%, 99%, or 100%) and/or is the amount sufficient to delay the manifestation, arrest the progression, relieve or alleviate at least one symptom of the demyelinating disorder as compared to no treatment.

### List of Abbreviations

CNS: central nervous system;
MS: multiple sclerosis;
OL: oligodendrocytes;
OPCs: oligodendrocyte precursor cells;
DIV: days in vitro;
RGC: retinal ganglion cell;
GSI: γ-secretase inhibitor;
MBP: myelin basic protein;
DAPT: N-[N-(3,5-Difluorophenacetyl)-L-alanyl]-S-phenylglycine t-butyl ester;
DMSO: dimethylsulfoxide;
HTS: high throughput screening;
NCC: NIH clinical collection.

### BRIEF DESCRIPTION OF THE FIGURES

FIG. 1 is a flow scheme illustrating the cortical cell myelination assay. A, Dissociated cells from the cortex containing neurons and glial progenitor cells were cultured from E18 rat embryos onto poly-D-lysine/laminin coated 96-well plates. B, On DIV4, when axonal projections (red) are apparent in the neuronal population, the growing co-culture is changed to MyM media to induce OL differentiation and initiate myelination. The following day test compounds are added and cultures are left undisturbed for an additional eight days. C, Cells are fixed and immunostained for MBP, Olig2 and DAPI on DIV13. Images were acquired using automated microscopy and scored phenotypically for myelination as described in the methods.

FIG. 2 illustrates oligodendrocyte processes align with cortical axons and γ-secretase inhibitors (GSIs) facilitate myelination. Cortical co-cultures were treated with the GSI, DAPT or DMSO as described above with respect to FIG. 1. A, On DIV13, cells were fixed and stained with antibodies to the axon marker SMI 31/32 neurofilament protein (red) and MBP (green).

Image at the right is a composite of the SMI 31/32, MBP, and DAPI. Arrowheads indicate regions of MBP alignment with axon. Bar=100 μm. B, Left two panels show entire image fields taken from a 96-well plate immunostained for Olig2 and MBP. Bars=200 μm. Boxed regions are enlarged in the middle panel to show morphological detail of MBP-stained OLs. Bar=50 μm. Two images at right depict the digital mask of MBP staining intensity of the adjacent image (middle panel) and the far right image are tracings of MBP alignment used to calculate fiber length. Bars=50 μm. C, Raw data from three DAPT dose response experiments was quantified from images as in B and compiled from n=3 experiments, 80 image fields per concentration, mean±SEM. Asterisk (*) denotes P values versus DMSO of <0.0001; ANOVA analysis, followed by Bonferroni correction.

FIG. 3 illustrates half maximal effective concentration determination of four different GSIs for the promotion of myelination in the cortical culture assay. Dose response data confirm the activity of GSIs and enable the calculation of the EC₅₀ value for each compound. Cortical cultures were treated for eight days with DAPT, LY 411,575, BMS 708,163 or MRK 560 and immunostained for MBP, Olig2 and DAPI. Dose-response curve for DAPT is compiled from n=3 experiments, 80 image fields per concentration. Representative dose-response curves for LY 411,575, BMS 708,163 and MRK 560 are 32 image fields per concentration, mean±SEM. Respective EC₅₀ values are shown in the legend.

FIG. 4 illustrates long term cortical cultures and demonstrates persistent GSI-induced enhancement of myelination and initiation of axonal node of Ranvier formation. On DIV5, cortical cultures were treated with DAPT or DMSO for eight days, media was changed weekly thereafter without compound, and cells fixed on DIV28. A, Left panels show triple immunostaining of MBP (green), Olig2 (red), and DAPI (blue). Red overlaid with blue appears pink. Right panels show digital masks created from MBP-stained images in the center panel. Masks were used for quantification of fiber length. Bars=100 μm. Arrows indicate areas with significant myelination. B, Quantification of myelination showing raw data in 28 DIV cortical cultures as in A. Representative data shown is averaged from 16 image fields per concentration, mean±SEM. Asterisk (*) denotes P values versus DMSO of <0.0001; ANOVA analysis, followed by Dunnett's correction. C, Cortical co-cultures were grown for a total of 21 days, fixed, and immunostained for MBP (green, merged image) and the paranode-localized protein Caspr (red, merged image). Note the accumulation of Caspr protein at the edges of myelinated axon segments (arrows). Bar, upper panels=100 μm. Bar, lower panels=50 μm.

FIG. 5 illustrates analysis of the cortical myelination screen of the NCC compound library. A, High-throughput screening data set used to identify promoters of myelination. The mean response is indicated by the solid line. The dotted line delineates the value of three SDs above the mean. Compounds that significantly reduced Olig2 expression were excluded. B, High-throughput screening data plate control values of myelination. Each point is a compiled control value from each screening plate (n=44, 16 image fields per concentration, mean±SEM*P<0.0001, t-test). C, Using the Fiber/MBP score as a specific measure of myelination (See FIG. S10), the ratio of the DAPT to DMSO controls demonstrates the screening assay window. The red line delineates the cutoff value of 1.3. Each point is an averaged value from each screening plate (32 image fields per condition, mean±SEM). The average DAPT/DMSO-Fiber/MBP ratio for the entire NCC library screen=1.61 (dashed line). D, NCC library hit selection process in the cortical culture myelination assay. Fifty three primary hits compounds were initially identified from the NCC library with the criteria of >50% DAPT and >1.5 Fiber/MBP ratio. The primary hits were further refined with additional criteria of >25% DAPT/Olig2 nuclei ratio, <40% DAPI/Olig2 nuclei ratio, and a visual morphology check to yield refined hits of 33 compounds. All refined hit compounds were reordered fresh and tested for efficacy in a dose-response profile. Ten compounds passed these criteria and were confirmed as hits.

FIG. 6 illustrates determination of embryonic cortical cultures for screening suitability. A, Myelination quantification of DMSO and DAPT control values were compared in two types of myelination culture preparations. Data shown was compiled from n=6 experiments, 32 image fields per test condition, mean±SEM. P values versus DMSO were determined by two-tailed t-test. Coefficient of variation (CV) values are reported below the graphs. CV values <20% were considered in the acceptable range. B, Schematic of the cortical co-culture preparation that demonstrates that three embryonic brains used for the cortical co-culture myelination assay will yield approximately fifty 96-well plates.

FIG. 7 illustrates the addition of exogenous OPCs to embryonic cortical cultures is not required for quantitative myelination. The promotion of myelination with DAPT was more robust (1.76 fold over DMSO) in cultures without exogenously added OPCs. The asterisk (*) denotes P values versus DMSO of <0.0001, t-test. A table of mean, standard deviation (SO), standard error of mean (SEM) and coefficient of variation (CV) values are reported below columns (64 image fields per treatment, mean±SEM). CV values of 20%+5% were considered in the acceptable range.

FIG. 8 illustrates determination of optimal time courses for myelination in the cortical cell myelination assay. E18 cortical cultures were initially differentiated for either 4 days (green bars), 5 days (black bars), or 6 days (blue bars) in NB/N21 media, followed by 4, 5, 6, 7, or 8 days in MyM, then fixed for antibody staining and image analysis. Numbers in bars indicate the DAPT/DMSO myelination ratio for each condition. The ratio values were compiled from 64 image fields, mean±SEM. The time course with greatest DAPT/DMSO myelination ratio was 5 days NB/N21 and 8 days MyM plus test compound (ratio=7.7) and was used in all subsequent assay development and screening.

FIG. 9 illustrates γ-secretase inhibitors do not promote OL differentiation, whereas benztropine and clemastine facilitate OL differentiation in an OL differentiation assay with acutely purified OPCs. Acutely prepared OPCs were cultured for 4 days (see methods) in the presence of increasing concentrations of test compound. 0.1% DMSO and 40 ng/ml T3 serve as negative and positive controls, respectively. Representative data shown are averaged from eight image fields per test concentration, mean±SEM. * denotes P values versus DMSO of <0.0001, ANOVA, followed by Bonferroni correction.

FIG. 10 illustrates benztropine and clemastine show little to no activity in the cortical myelination assay. Dose response experiments were performed adding test compound to cortical cultures on DIVS and incubated for an additional eight days as described above. Representative raw data is averaged from 16 image fields per concentration, mean±SEM.

FIG. 11 illustrates neuronal characterization of DIV13 cortical cultures. Control cortical cultures were treated with 0.1% DMSO on DIVS, then on DIV13, fixed and stained with the antibodies labeled in the left panels. Right images are merged from left and middle panels with antibody staining in red and DAPI staining in blue, overlapping staining appears pink. Counting NeuN, Olig2, and GFAP positive cells with overlapping DAPI staining, these cultures were calculated to have approximately 22.5% neurons, 22% OPCs/OLs, and 46% astrocytes. Bar=200 μm.

FIG. 12 illustrates neuronal characterization of DIVS cortical cultures. Cortical cultures were established and grown until DIV5, fixed and stained with the antibodies labeled in the left panels. Right images are merged from left and middle panels with antibody staining in red and DAPI staining in blue, overlapping staining appears pink. Bar=200 μm.

FIG. 13 illustrates A2B5 marker antibodies identify abundant glial progenitor cells in DIV5 cortical cultures, but are largely absent in DIV13 cultures. Cortical cultures were grown, fixed and stained with anti-A2B5 antibodies on either DIV5 (day of test compound addition) or DIV13 (endpoint of myelination assay). Images at the right show the merged images of A2B5 (red) and DAPI (blue). Note the almost complete absence of A2B5 staining in the DIV13 cultures. Bar=200 μm.

FIG. 14 illustrates oligodendrocyte characterization of DIV5 and DIV13 cortical cultures, demonstrate robust OL differentiation during the test compound treatment window. Cortical cultures were grown, fixed and stained with the antibodies labeled at the left. Note the robust expression of OL markers in the DIV13 cultures. Bar=200 μm.

FIG. 15 illustrates equations for the quantification of myelination. Schematic figure defining the image quantification calculations derived from MBP intensity mask and number of Olig2 positive cells. OL differentiation is total MBP intensity/Olig2 nuclei and early myelination is calculated as the total length of contiguous MBP staining (fiber length)/Olig2 nuclei. The fiber length/MBP intensity ratio is a score that normalizes the OL differentiation contribution revealing morphological changes specific to MBP alignment with axons.

FIG. 16 illustrates structures, images, and EC₅₀ curves of cortical myelination and OL differentiation hits. A, Chemical structure and name of each hit compound with the controls, 0.1% DMSO and 1 IJM DAPT. B, Example image of each compound directly from the library screening plate at the most efficacious concentration showing MBP (green), Olig2 (red) and DAPI (blue) staining. Olig2 overlapping with DAPI staining appears pink. Bar=200 IJM. C, Enlarged monochrome image (stained for MBP) of each hit from the library screen to highlight OL morphological changes. Bar=50 μM. D, Representative myelination dose-response curves of each hit (D).

### DETAILED DESCRIPTION

The present invention relates to treatment of demyelinating disorders. Specifically, the present invention relates to methods of treatment using one or more therapeutics which promote myelination alone or in combination with other therapeutics for the treatment of demyelinating disorders.

In certain embodiments of the present invention, therapeutics which promote myelination were identified using a high throughput in vitro cortical myelination assay. In specific embodiments the cortical cell myelination assay method comprises: (a) culturing dissociated cells from a sample cortex containing neurons and glial progenitor cells in a first culture media to produce a neuronal media; (b) inducing oligodendrocyte differentiation and initiating myelination when axonal projections are apparent in the neuronal cell population by replacing the first culture media with a second culture media; (c) introducing a test compound to the neuronal cell population in the second culture media and incubate for a period of time; (d) fixing and staining cells of incubated neuronal cell population; (e) imaging fixed and stained cells; and (f) scoring cells phenotypically for myelination.

The assay can be utilized to screen novel therapeutics or known therapeutics for promyelination activity. Libraries of potential therapeutics can be screened using the myelination assay to identify therapeutics exhibiting promyelinating activity. The libraries can include novel and/or known therapeutics. A non-limiting example of a library comprising known small molecules is the NIH Clinical Collection library.

Therapeutics which were identified using the described high throughput in vitro cortical myelination assay as exhibiting promyelinating activity include but are not limited to TRPV1 agonists. TRPV1 is the transient receptor potential cation channel subfamily V member 1 (TrpV1), also known as the capsaicin receptor and the vanilloid receptor 1. TRPV1 is found in both the peripheral nervous system and central nervous system.

Non-limiting examples of TRPV1 agonists include but are not limited to zu-capsaicin (i.e. cis-capsaicin, Civanex™); capsaicin; cannabinoids (see, for example, Costa et al., 34 and lannotti et al., 35) including but not limited to cannabidivarin and cannabidiol; endocannabinoids including but not limited to anadamide (N-arachidonoyl ethanolamine) and N-Arachidonoyl dopamine; vanilloids; resiniferatoxin; AM-404 [N-(4-hydroxyphenyl)-arachidonoyl-ethanolamine]; N-acyl ethanolamines (NAEs); N-oleoylethanolamine (OLEA); N-oleoyl dopamine (OLDA); 5-(S), 8-(S), 12-(S) and 15-(S)-hydroperoxyeicosatetraenoic acids (HPETEs); hepoxilins A3 (HXA3); ATP; spermine; spermidine; putrescine; 13(S)-hydroxy-9Z,11E-octadecadienoic acid (13(S)-HODE); 13(R)-hydroxy-9Z,11E-octadecadienoic acid (13(R)-HODE); 9(S)-hydroxy-10(E); 12(Z)-octadecadienoic acid (9(S)-HODE); 9(R)-hydroxy-10(E); 12(Z)-octadecadienoic acid (9(R)-HODE); 13-oxoODE; 9-oxoODE; 20-hydroxy-5Z,8Z,11Z,14Z-eicosatetraenoic acid; 12(S)-hydroxy-5Z,8Z,10E,12S,14Z-eicosatetraenoic acid (12(S)-HETE, hepoxilin A3 (i.e. 8R/S-hydroxy-11,12-oxido-5Z,9E,14Z-eicosatrienoic acid) and H×B3 (i.e. 10R/S-hydroxy-11,12-oxido-5Z,8Z,14Z-eicosatrienoic acid).

Accordingly, in certain embodiments, there is provided a method of repairing and/or maintaining the myelin sheath of neuronal axons in a subject comprising administering an effective amount of one or more TRPV1 agonists exhibiting promyelinating activity alone or in combination with other therapeutics. In specific embodiments, the one or more TRPV1 agonists are selected from the group consisting of zu-capsaicin, capsaicin, cannabinoids, such as cannabidivarin and cannabidiol, anadamide, vanilloids and combination thereof. In particular embodiments, the method comprises administering an effective amount of zu-capsaicin. Also provided are compositions comprising the one or more TRPV1 agonists for repairing and/or maintaining the myelin sheath of neuronal axons, including in specific embodiments, compositions comprising zu-capsaicin for repairing and/or maintaining.

In certain embodiments, there is provided a method of promoting myelination of an axon of a nerve cell comprising contacting the nerve cell with an effective amount of one or more TRPV1 agonists. Also provided are compositions comprising one or more TRPV1 agonists for promoting myelination of an axon of a nerve cell. In specific embodiments, the one or more TRPV1 agonists are selected from the group consisting of zu-capsaicin, capsaicin, cannabinoids, such as cannabidivarin and cannabidiol, anadamide, vanilloids and combination thereof.

In certain embodiments, there is provided a method of treating a demyelinating disorder in a subject, said method comprising administering an effective amount of one or more TRPV1 agonists exhibiting promyelinating activity alone or in combination with other therapeutics. In specific embodiments, the one or more TRPV1 agonists are selected from the group consisting of zu-capsaicin, capsaicin, cannabinoids, such as cannabidivarin and cannabidiol, anadamide, vanilloids and combination thereof. In particular embodiments, the method comprises administering an effective amount of zu-capsaicin. Also provided are compositions comprising the one or more TRPV1 agonists for treating a demyelinating disorder in a subject.

Optionally, the one or more TRPV1 agonists exhibiting promyelinating activity can be used in combination with various other treatments which can be useful for the treatment of demyelinating disorders. Other therapeutics include but are not limited to anti-inflammatory agents, immune modulators, other agents having promyelinating activity or remyelination agents and known therapies for treatment of the demyelinating disorders. For example, one or more TRPV1 agonists can be administered in combination with at least one of interferon beta 1a, interferon beta 1b, glatiramer acetate, mitoxantrone, azathiprine, cyclophosphamide, cyclosporine, ampyra, dimethyl fumarate, fingolimod, methotrexate, cladribine, methylprednisone, prednisone, prednisolone, dexamethasone, adreno-corticotrophic hormone, Corticotropin, anti-integrin specific antibodies, cytoxan, naltrexone, and the like. The one or more TRPV1 agonists can be also administered in combination with anti-LINGO therapies, axin/Wnt pathway inhibitors, and/or agonists for RXR transcription factors such as, e.g., 9-cis-retinoic acid.

The demyelinating disorders that may be treated by the methods of the invention include demyelinating disorders of the central nervous system (CNS) and/or peripheral nervous system (PNS), demyelinating injuries that occur as a result of specific or focal insults such as stroke or traumatic brain injury, or degradation that may be progressive in nature and associated with normal cognitive or physical decline with age. The demyelinating disorders may include inflammatory demyelinating disorders and non-inflammatory demyelinating disorders. Many demyelinating disorders are classified as either myelinoclastic or leukodystrophic.

Exemplary demyelinating disorders of the central nervous system include but are not limited to multiple sclerosis; Devic's disease (neuromyelitis optica); other inflammatory demyelinating diseases such as acute-disseminated encephalomyelitis and acute haemorrhagic leucoencephalitis; demyelinating disease precipitated by tumor necrosis factor alpha antagonists or other immunomodulators; viral demyelinating diseases such as progressive multifocal leukoencephalopathy and Tabes dorsalis; acquired metabolic demyelination diseases such as central pontine myelinolysis and extrapontine myelinolysis; hypoxic-ischaemic demyelination, compression-induced demyelination and leukodystrophies including but not limited to Adrenomyeloneuropathy, Alexander disease, Cerebrotendineous xanthomatosis, Hereditary CNS demyelinating disease, Krabbe disease, Metachromatic leukodystrophy, Pelizaeus-Merzbacher disease, Canavan disease, leukoencephalopathy with vanishing white matter, Adrenoleukodystrophy and Refsum disease.

Exemplary demyelinating disorders of the peripheral nervous system include but are not limited to Guillain-Barré syndrome; chronic inflammatory demyelinating polyneuropathy; Anti-MAG peripheral neuropathy; Charcot-Marie-Tooth disease; copper deficiency associated conditions and progressive inflammatory neuropathy.

Exemplary demyelinating disorders involving both the central nervous system and peripheral nervous system include but are not limited to acute combined central and peripheral inflammatory demyelination.

In certain embodiments, the methods of the invention treat demyelinating disorders of the CNS in a subject. In specific embodiments, the methods of the invention treat multiple sclerosis in a subject. Also provided in certain embodiments are compositions comprising one or more TRPV1 agonists exhibiting promyelinating activity alone or in combination with other therapeutics for use in the treatment of a demyelinating disorder of the CNS, including but not limited to multiple sclerosis. Accordingly, in some embodiments the compositions of the invention are specifically formulated for treatment of CNS diseases or for administration to the CNS.

In certain embodiments, the methods of the invention treat demyelinating disorders of the PNS in a subject. Also provided in certain embodiments are compositions comprising one or more TRPV1 agonists exhibiting promyelinating activity alone or in combination with other therapeutics for use in the treatment of a demyelinating disorder of the PNS in a subject. Accordingly, in some embodiments the compositions of the invention are specifically formulated for treatment of PNS diseases or for administration to the PNS.

In certain embodiments, the methods of the invention treat demyelinating disorders of the CNS and PNS in a subject. Also provided in certain embodiments are compositions comprising one or more TRPV1 agonists exhibiting promyelinating activity alone or in combination with other therapeutics for use in the treatment of a demyelinating disorder of the CNS and PNS in a subject.

Remyelination of demyelinated axons may be neuroprotective. Accordingly, in certain embodiments, there is provided a method of neuroprotection comprising administering to a subject an effective amount of one or more TRPV1 agonists exhibiting promyelinating activity alone or in combination with other therapeutics.

In certain embodiments, the composition comprising the one or more TRPV1 agonists and optionally other therapeutics further comprise a pharmaceutically acceptable carrier. Pharmaceutically acceptable carriers include, for example, pharmaceutically acceptable solvents, suspending agents, or any other pharmacologically inert vehicles. Pharmaceutically acceptable carriers can be liquid or solid, and can be selected with the planned manner of administration in mind so as to provide for the desired bulk, consistency, and other pertinent transport and chemical properties, when combined with one or more therapeutic compounds and any other components of a given pharmaceutical composition. Typical pharmaceutically acceptable carriers include, without limitation: water; saline solution; binding agents (e.g., polyvinylpyrrolidone or hydroxypropyl methylcellulose); fillers (e.g., lactose or dextrose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

Pharmaceutical compositions of the invention can be administered by a number of methods, depending upon whether local or systemic treatment is desired. Administration can be, for example, parenteral (e.g., by subcutaneous, intrathecal, intraventricular, intramuscular, or intraperitoneal injection, or by intravenous (i.v.) drip); oral; topical (e.g., transdermal, sublingual, ophthalmic, or intranasal); or pulmonary (e.g., by inhalation or insufflation of powders or aerosols), or can occur by a combination of such methods. Administration can be rapid (e.g., by injection) or can occur over a period of time (e.g., by slow infusion or administration of slow release formulations).

In certain embodiments, there is provided a pharmaceutical composition having promyelinating activity comprising one or more TRPV1 agonists selected from the group consisting of zu-capsaicin, capsaicin, cannabinoids, such as cannabidivarin and cannabidiol, anadamide, vanilloids and combination thereof. In specific embodiments, there is provided a pharmaceutical composition comprising zu-capsaicin formulated for intranasal or intrathecal injection.

Compositions and formulations for parenteral, intrathecal or intraventricular administration may include sterile aqueous solutions (e.g., sterile physiological saline), which also can contain buffers, diluents and other suitable additives (e.g., penetration enhancers, carrier compounds and other pharmaceutically acceptable carriers).

Compositions and formulations for oral administration may include, for example, powders or granules, suspensions or solutions in water or non-aqueous media, capsules, sachets, or tablets. Such compositions also may incorporate thickeners, flavoring agents, diluents, emulsifiers, dispersing aids, or binders.

Formulations for topical administration may include, for example, sterile and non-sterile aqueous solutions, non-aqueous solutions in common solvents such as alcohols, or solutions in liquid or solid oil bases. Such solutions also may contain buffers, diluents and other suitable additives. Pharmaceutical compositions and formulations for topical administration can include transdermal patches, ointments, lotions, creams, gels, drops, suppositories, sprays, liquids, and powders. Conventional pharmaceutical carriers, aqueous, powder or oily bases, thickeners and the like may be useful. Methods and compositions for transdermal delivery may include those described in the art (e.g., in Wermeling et al. (2008) Proc. Natl. Acad. Sci. USA 105:2058-2063; Goebel and Neubert (2008) Skin Pharmacol. Physiol. 21:3-9; Banga (2007) Pharm. Res. 24:1357-1359; Malik et al. (2007) Curr. Drug Deliv. 4:141-151; and Prausnitz (2006) Nat. Biotechnol. 24:416-417).

Nasal preparations may be presented in a liquid form or as a dry product. Nebulized aqueous suspensions or solutions can include carriers or excipients to adjust pH and/or tonicity.

Pharmaceutical compositions include, but are not limited to, solutions, emulsions, aqueous suspensions, and liposome-containing formulations. These compositions can be generated from a variety of components that include, for example, preformed liquids, self-emulsifying solids and self-emulsifying semisolids.

Compositions additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions, such as dyes, flavoring agents, preservatives, antioxidants, opacifiers, thickening agents, and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, penetration enhancers, and aromatic substances. When added, however, such materials should not unduly interfere with the biological activities of the other components within the compositions.

In some cases, the one or more TRPV1 agonists and optionally other therapeutics can be formulated as a sustained release dosage form, or within pharmaceutical prodrug formulations that enable the conversion of the prodrug into the active TRPV1 agonists within the body upon administration.

Pharmaceutical formulations as disclosed herein, which can be presented conveniently in unit dosage form, can be prepared according to conventional techniques well known in the pharmaceutical industry. Such techniques include the step of bringing into association the active ingredient(s) (i.e., the one or more TRPV1 agonists and optionally other therapeutics) with the desired pharmaceutical carrier(s). Typically, the formulations can be prepared by uniformly and intimately bringing the active ingredient(s) into association with liquid carriers or finely divided solid carriers or both, and then, if necessary, shaping the product. Formulations can be sterilized if desired, provided that the method of sterilization does not interfere with the effectiveness of the molecules(s) contained in the formulation.

The compositions of the invention may further comprise agents which facilitate brain delivery. Non-limiting examples of such useful agents include, e.g., an implantable reservoir (Omaya reservoir), functionalized nanocarriers and liposomes.

The following examples illustrate embodiments of the invention, but should not be viewed as limiting the scope of the invention.

EXAMPLES

### Introduction

A high throughput myelination assay was developed and utilized to identify potential myelin repair therapeutics. In an effort to create a myelination assay more amenable to higher throughput compound screening, embryonic rat cortex was used to develop, optimize, and validate an in vitro myelination assay [5, 6] which may be utilized for chemical library screening. The culture system was miniaturized into a 96-well plate format enabling high throughput liquid handling, automated image acquisition and analysis of myelinating co-cultures. It has previously been shown that inhibition of the γ-secretase protease activity promotes differentiation of OPCs and myelination of retinal ganglion cells (RGC) in RGC-OPC co-cultures. [7], [8], [9]. Based on this published work, the γ-secretase inhibitor (GSI), N-[N-(3,5-Difluorophenacetyl)-L-alanylFS-phenylglycine t-butyl ester (DAPT) was used as a positive control in the cortical co-cultures [9], and confirmed that the assay allows for the quantification of early axonal myelination in a dose-dependent manner. This assay identified compounds which are not active in a pure primary OPC differentiation assay [3, 10] but are capable of promoting re-myelination in vivo [11]. This myelination assay was used to screen the NIH clinical collection library of small molecules.

### Development of a High Throughput Cortical Myelination Assay

Focusing solely on the immunological component of MS only addresses one aspect of the disease. Repairing damaged myelin and/or promoting the remyelination of demyelinated axons within lesions would, at a minimum, facilitate the preservation and/or restoration of some neuronal function. This may also prevent the irreversible neuronal damage believed to underlie the progressive disability that eventually affects most MS patients. Thus, remyelinating compounds are highly sought after, but have been difficult to identify in part because of the lack of high throughput screening (HTS) assays that truly detect myelination.

The goal for developing a co-culture with live axons and oligodendrocytes as a myelinating in vitro system was to overcome the challenges of labor intensive OPC/neuron preparations, inconsistent performance of classical sources of neurons for modeling myelination (e.g. retinal ganglion cells (RGCs), dorsal root ganglion cells), and generating sufficient quantities of cells required for a robust HTS assay. In this study, an in vitro myelination assay that assesses the functional dynamic interaction between live axons and oligodendrocytes during myelination and can be performed with a simple culture technique at a scale and reproducibility amenable to HTS drug discovery was developed. This assay is unique in that it evaluates test compounds in the presence of the co-developing milieu of native brain cells, including oligodendrocytes (OLs), neurons, and astrocytes. It was demonstrated that primary embryonic cortical tissue is an abundant cell source for both neurons and oligodendrocyte precursor cells (OPCs) that are myelination competent [6] [5], easier to culture than RGCs, and widely used in large-scale HTS screening within the pharmaceutical industry. This assay was validated using γ-secretase inhibitors (GSIs), EC50 values for four different compounds was established to allow the ranking of potency. Using this assay, the NCC library was screened and ten confirmed hit compounds from diverse target classes for follow-up characterization were identified.

In the cortical myelination assay the OLs develop and differentiate alongside growing axons and astrocytes, two major sources of signaling molecules known to influence myelination. The expression of the axonal protein LRR and Ig domain-containing, Nogo receptor-interacting protein (LINGO-1) was demonstrated be a potent inhibitor of differentiation and myelination [15] [16, 17]. Indeed, anti-LINGO-1 antibody is being developed as an MS therapeutic to promote axon remyelination and is currently in human clinical trials (BIIB033, ClinicalTrials.gov identifiers: NCT01244139, NCT01052506, NCT01864148). Leukemia inhibitory factor (LIF) has been shown to be released by astrocytes in response to ATP from action potential firing axons to promote myelination [16]. Additionally, through the action of TNFR2 on astrocytes, LIF is produced to stimulate OL differentiation in a co-culture system [18]. Furthermore, astrocytes were demonstrated to reduce OL differentiation, but specifically enhance myelin thickness and the rate of axon wrapping [9]. TNF impairs OL differentiation [19] attenuating TNF signaling by TNFR1 blocking therapy ameliorates MS symptoms in EAE [20]. It was also demonstrated that inhibiting glial γ-secretase promoted myelination [9], which also showed efficacy in vivo in the EAE model of MS [11]. These observations emphasize the importance of having culture conditions that more closely mimic the in vivo CNS composition.

The assay could be modified by spiking in test compounds during the course of the ensheathment window and/or lengthen the ensheathment window longer than eight days. T3, forskolin, and CNTF was included in the MyM medium as factors that facilitate OL differentiation and survival [21]. The activity of these factors may mask effects of potential stimulators of myelination. In particular, elimination of T3, may lower the threshold for identifying additional candidate compounds. This stimulation of differentiation by T3 may account for the lack of OL differentiation activity of benztropine and clemastine in the cortical myelination assay. Elimination of these factors from the myelination phase of the assay may reveal additional compounds with myelination activity.

### The Cortical Myelination Assay Identifies Compounds Not Revealed by OL Differentiation Assays

The myelination assay described herewith greatly differs from in vitro OL differentiation assays used for compound screening which have only assessed differentiation using purified OPCs (in isolation from axons and astrocytes) adapted to culture conditions by multiple passages [3], [2], or differentiated from induced pluripotent stem cells [22] and carried out in very short developmental time frames. Mei et al., 2014 [2] developed an HTS assay incorporating OL differentiation in the presence of inert micropillers allowing the quantification of pillar wrapping as a surrogate for myelination [2]. Lead compounds identified from these three studies, including clemastine, benztropine, miconazole and clobetasol, facilitate OL differentiation in cultures of purified OPCs [3], [2], [4] (FIG. 12), but had no effect on myelination in the live axon myelination assay described here (FIG. 13, Table 2, compounds 450—clobetasol and 588—miconazole). Notably, the myelination assay did not identify muscarinic antagonists as previously identified by independent OL differentiation screens [3],[2], but revealed entirely new classes of compounds that promote myelination. No other high throughput assay has been developed to date capable of assessing the initiation and facilitation of myelination in the presence of axons, arguably the most important features when selecting candidate compounds capable of promoting or generating remyelination.

### Relation of Cortical Myelination Assay Hit Compounds to Clinical Applications and Multiple Sclerosis

Repositioning approved drugs for the treatment of new indications is an activity that has grown in popularity in recent years and is a trend that is predicted to continue. Eight out of ten confirmed hit compounds aligned with current MS repositioning efforts. The confirmed hits include: Digoxin (LANOXIN™), Imatinib mesylate (GLEEVEC), Artesunate, Methotrexate (TREXALL™), Oxcarbazepine (TRILEPTAL®) and docetaxel (TAXOTER®).

The remainder of the confirmed hits, zu-capsaicin (CIVANEX™) and tegafur (UFTORAL®) have not been previously tested in any demyelinating diseases. In particular, the myelin restorative effects of these compounds have not been previously evaluated. The data suggests that these drugs may have beneficial effects on remyelination in vivo and possibly in MS patients.

### Results

### Development of the Embryonic Cortical Cell Co-Culture Assay.

In an effort to move a low-throughput, well-established myelination cell culture technique to a format suitable for higher throughput screening applications, a previously described RGC-OPC co-culture technique [9] was miniaturized and automated. Unfortunately, the low yield of RGCs and lack of assay robustness from each preparation makes this co-culture myelination assay unsuited for high-throughput compound screening (FIG. 6A). Another source of tissue where numbers of neurons would not be limiting was sought.

For increased yield of primary neurons, the cortex of embryonic day 18 (E18) rats was chosen as an abundant source of relatively homogeneous brain cells with well-established culture methods [5, 6] (see methods). From one litter, enough cells can easily be generated for high throughput drug screening applications (˜30×10⁶ cells/cortex; FIG. 6B).

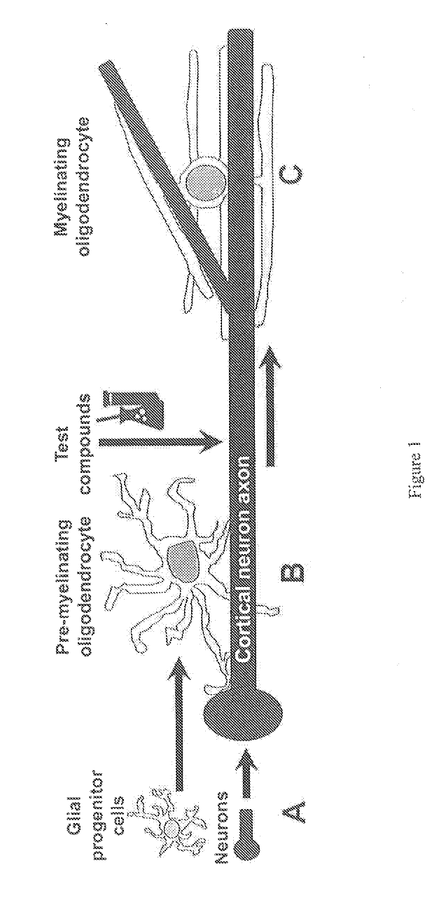

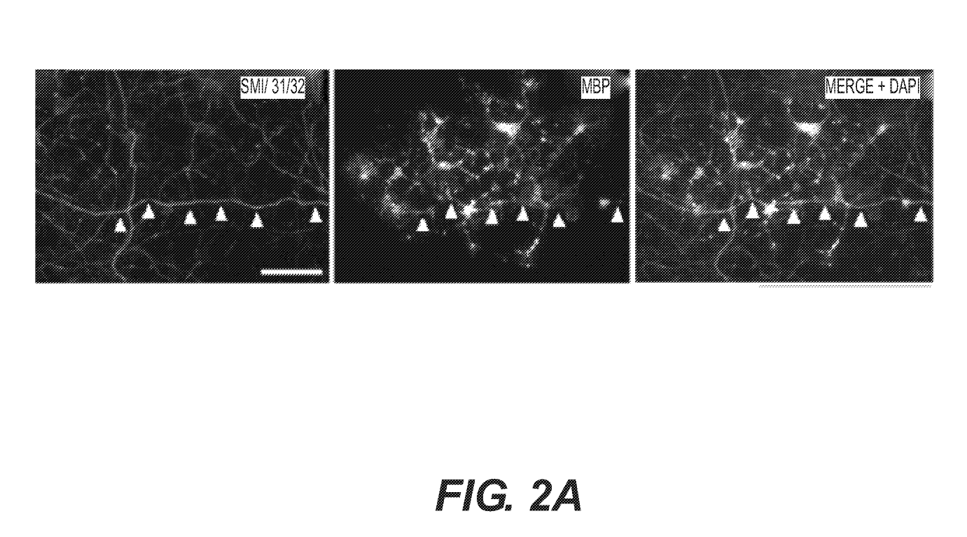

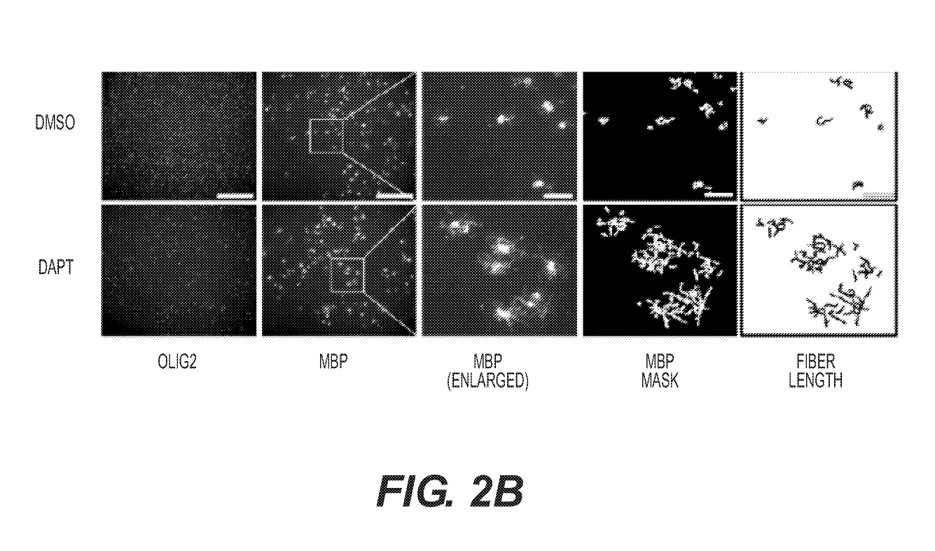

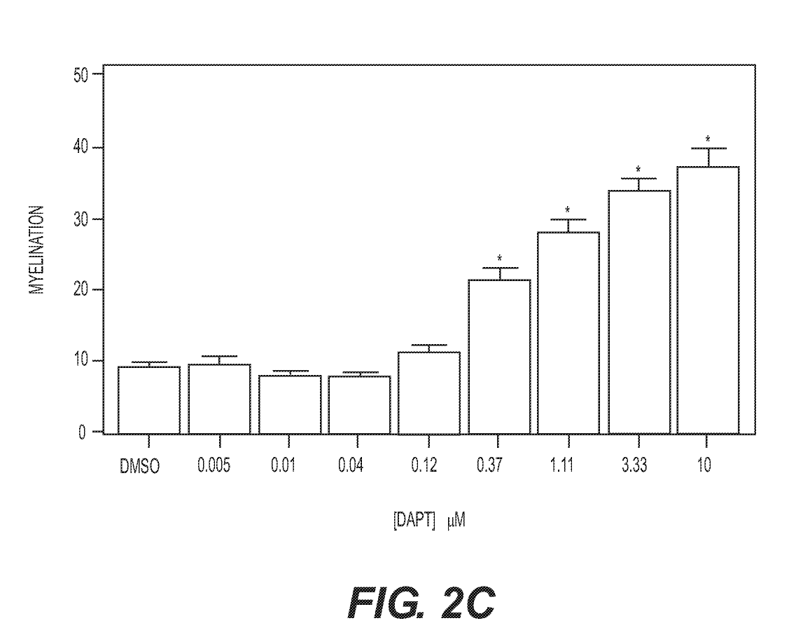

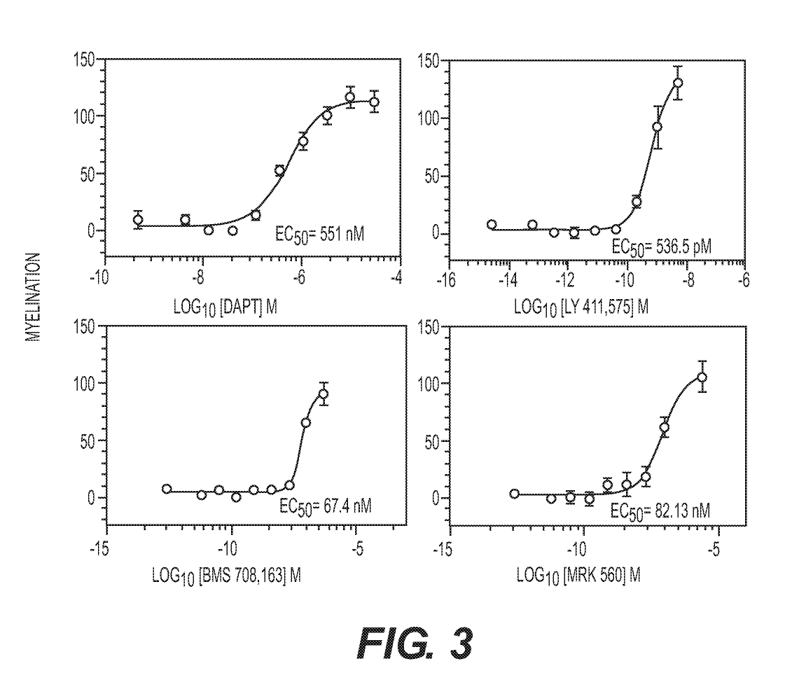

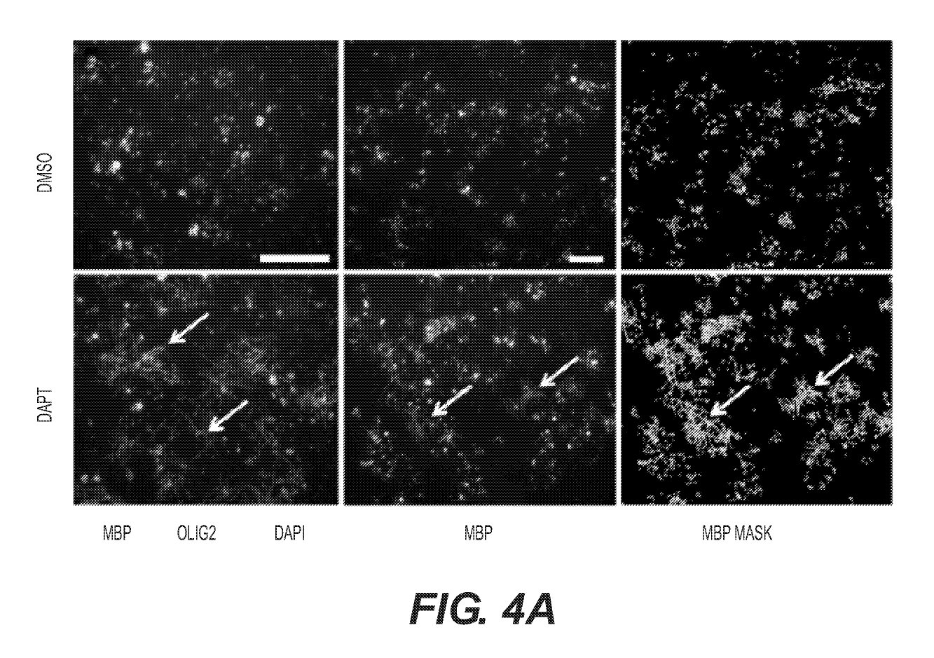

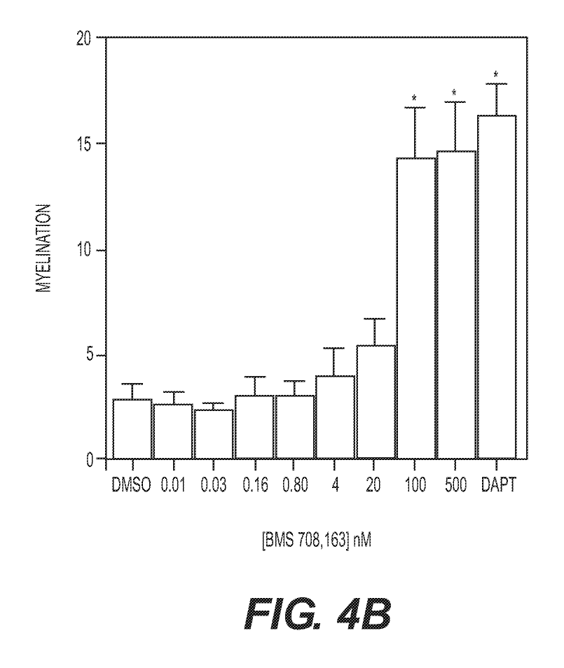

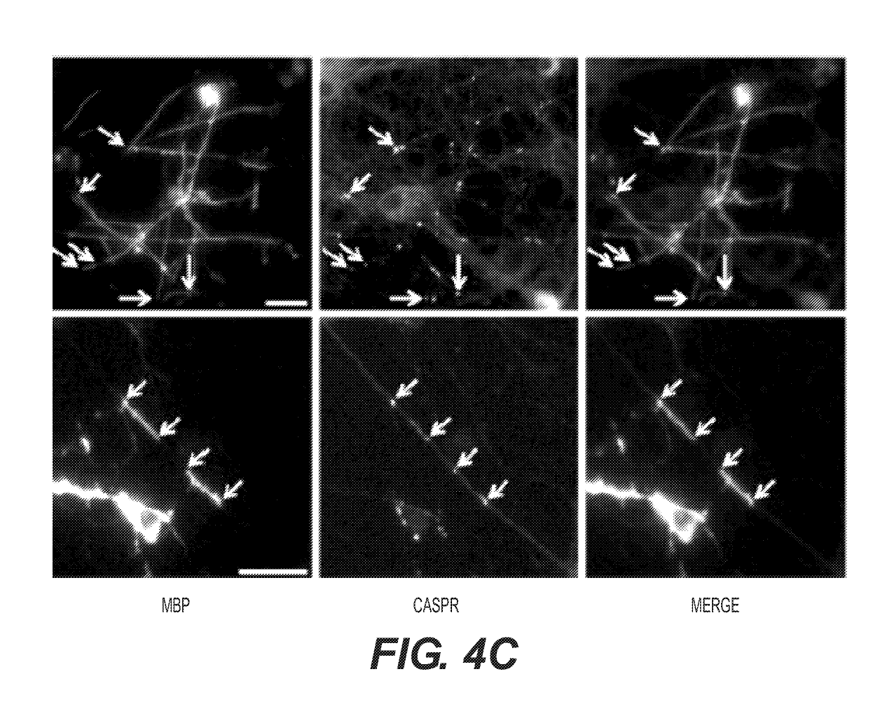

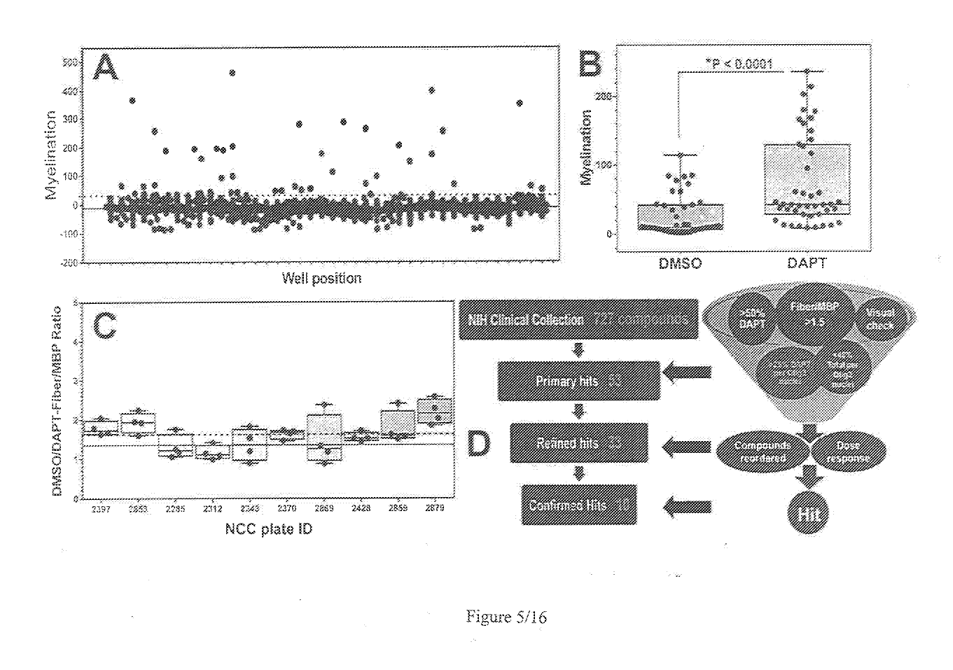

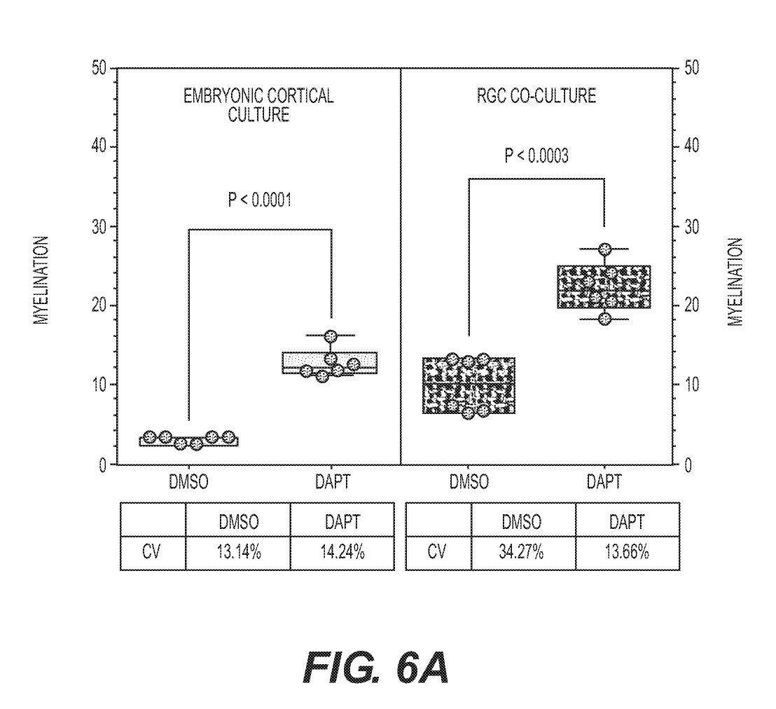

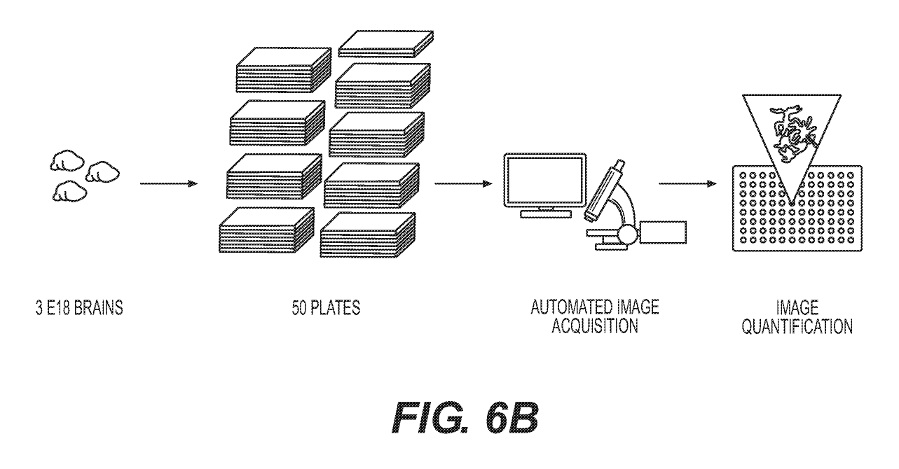

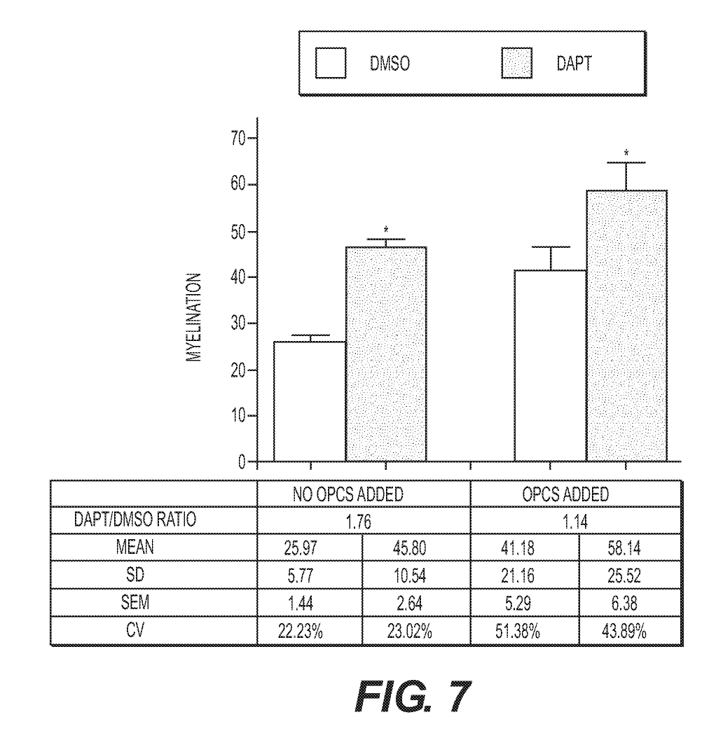

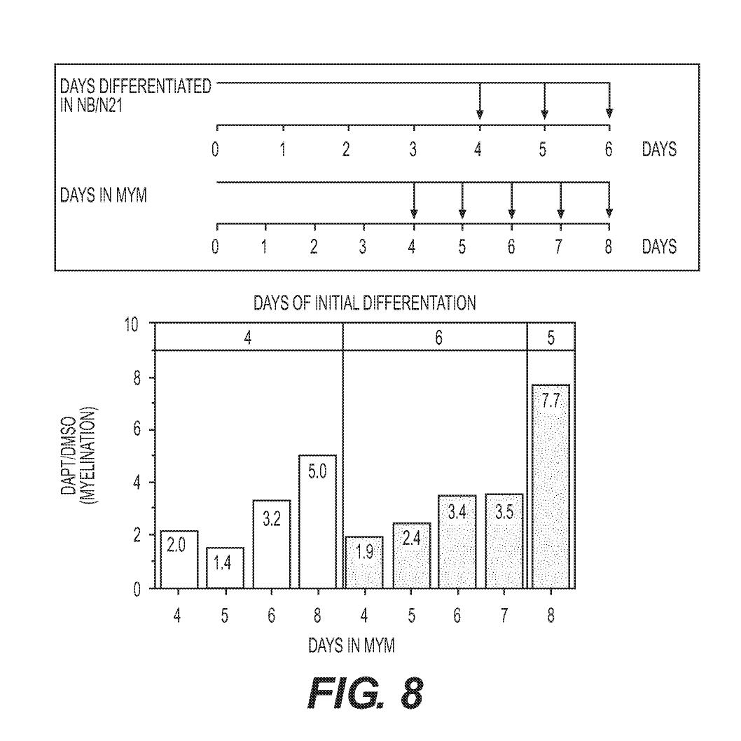

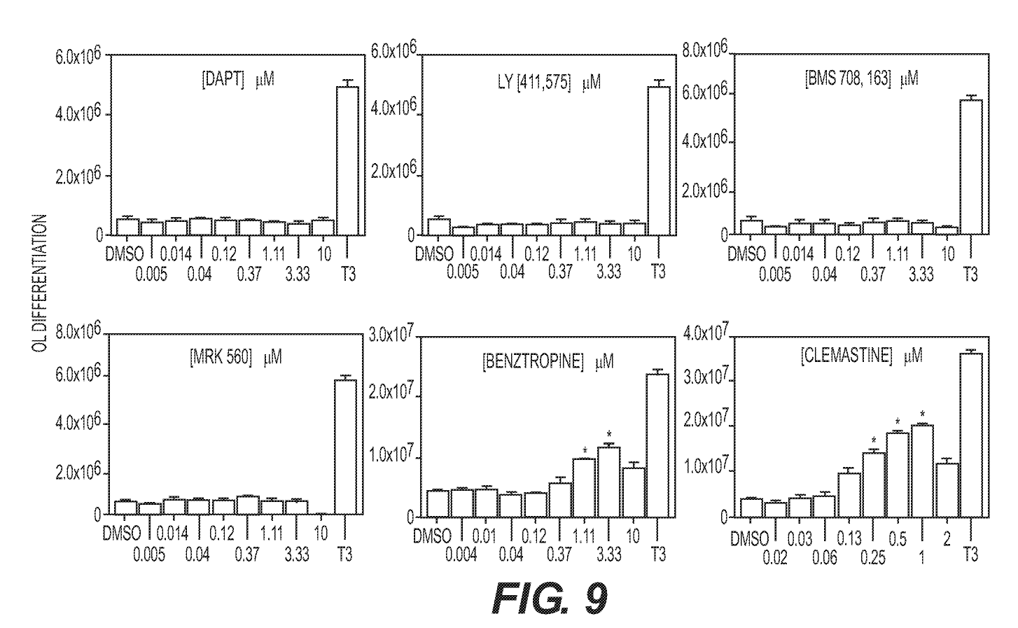

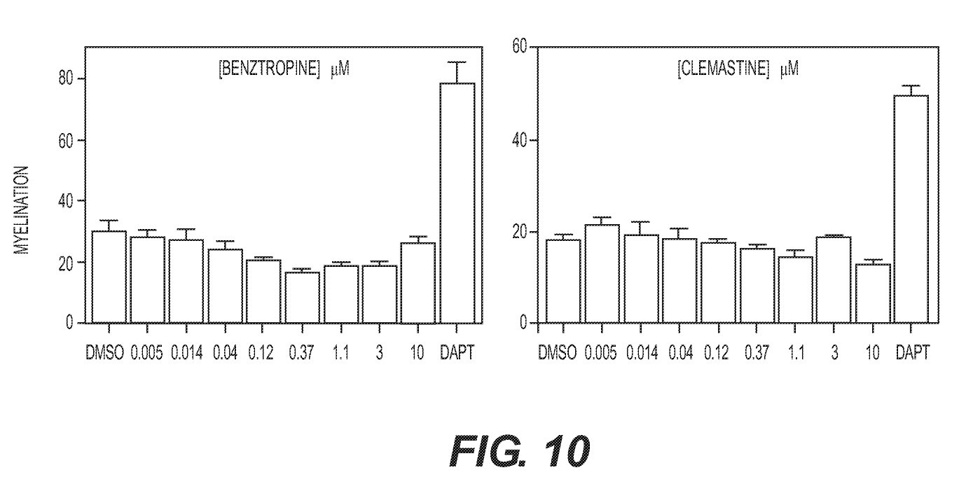

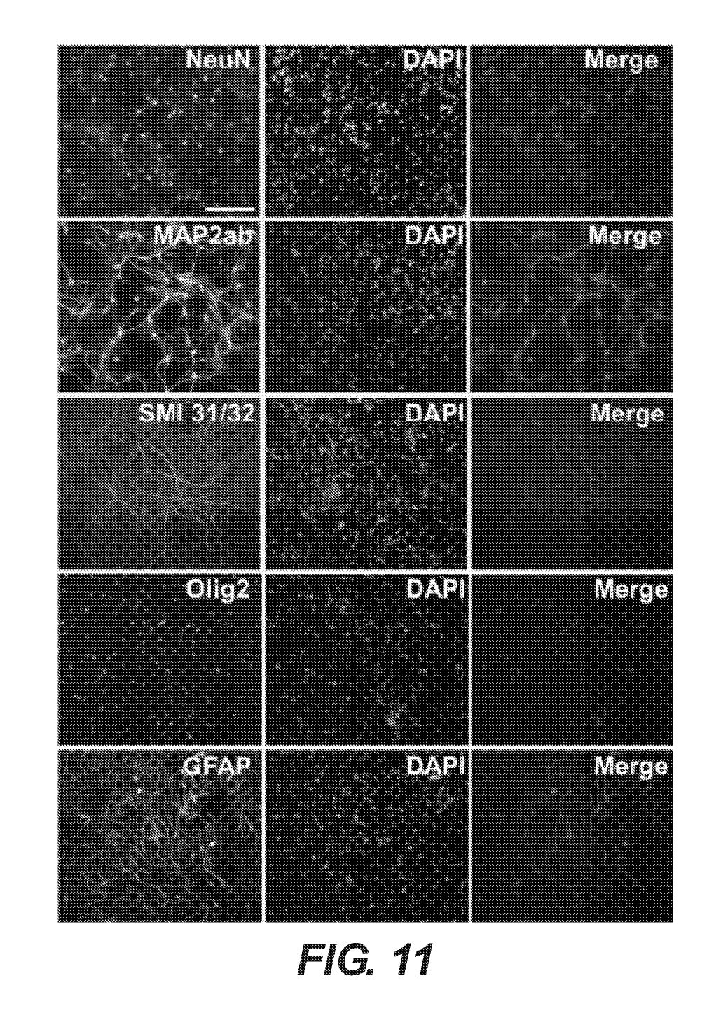

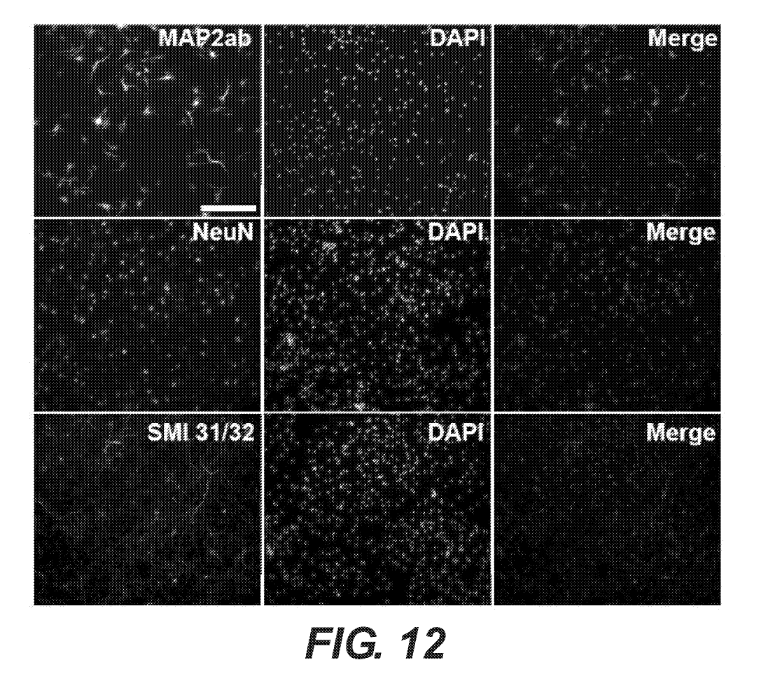

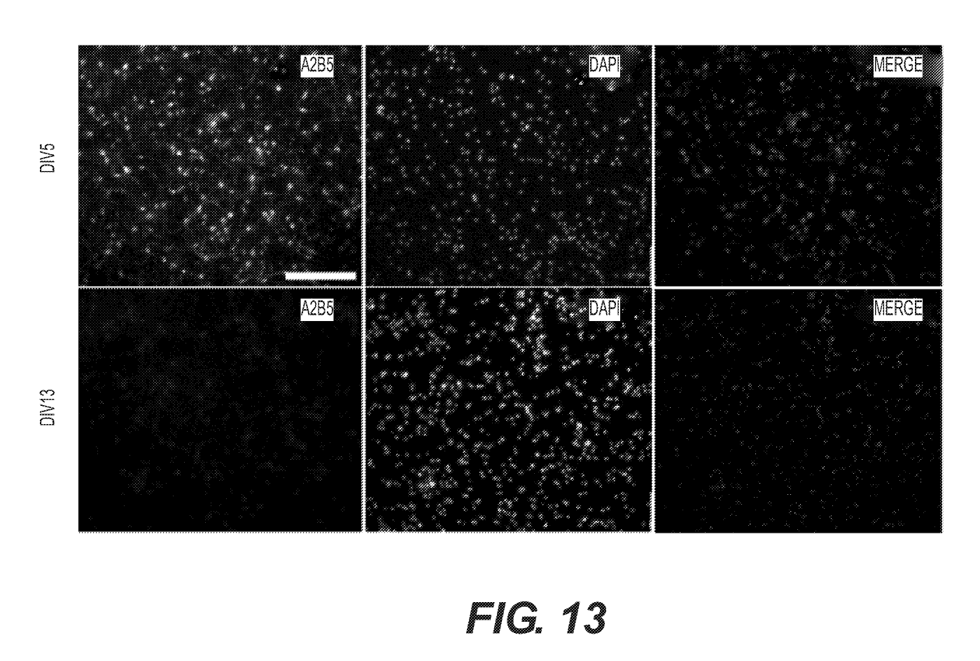

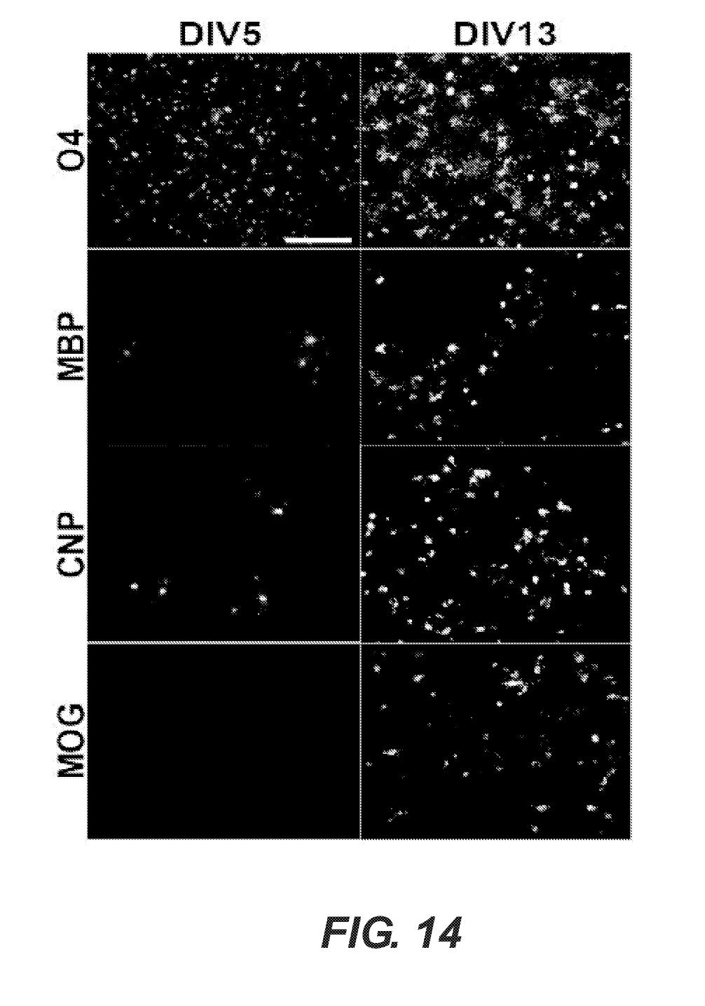

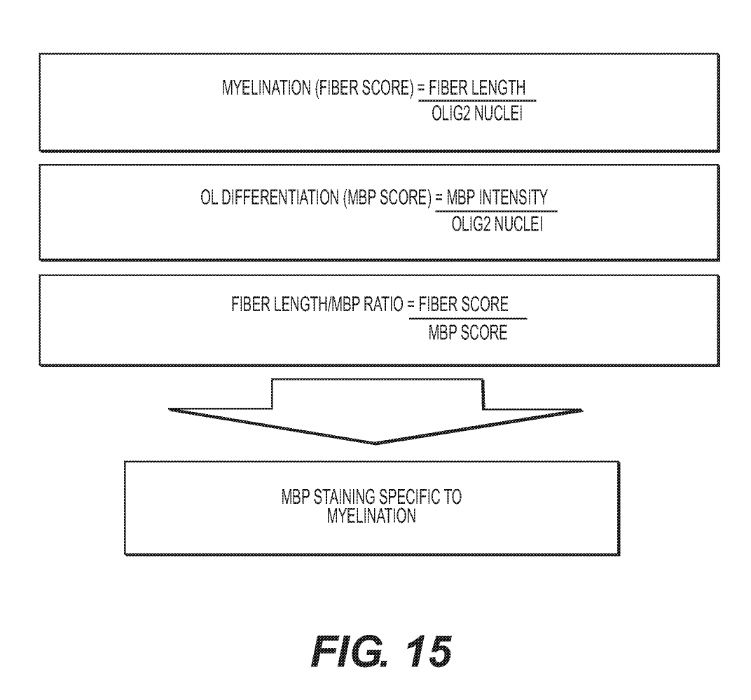

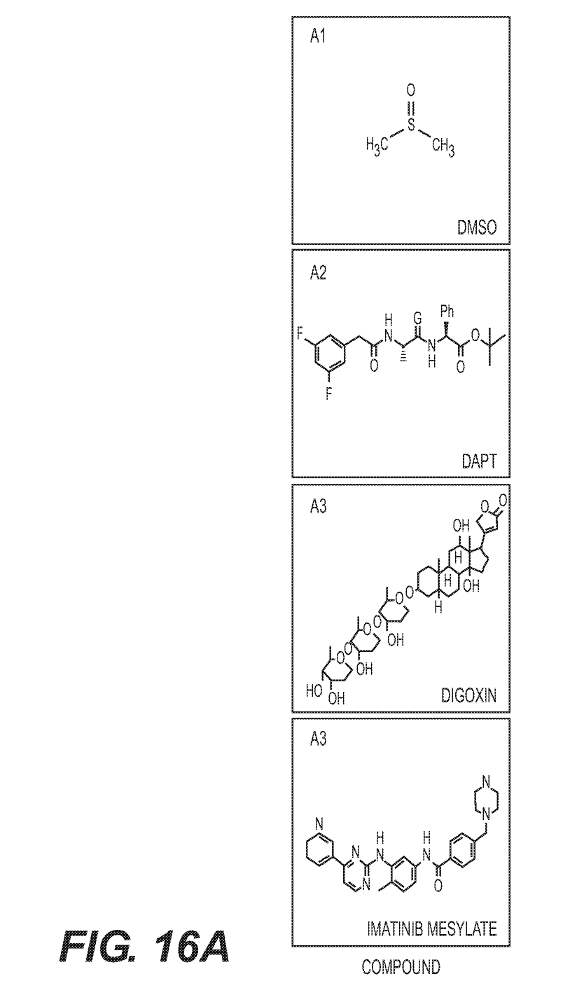

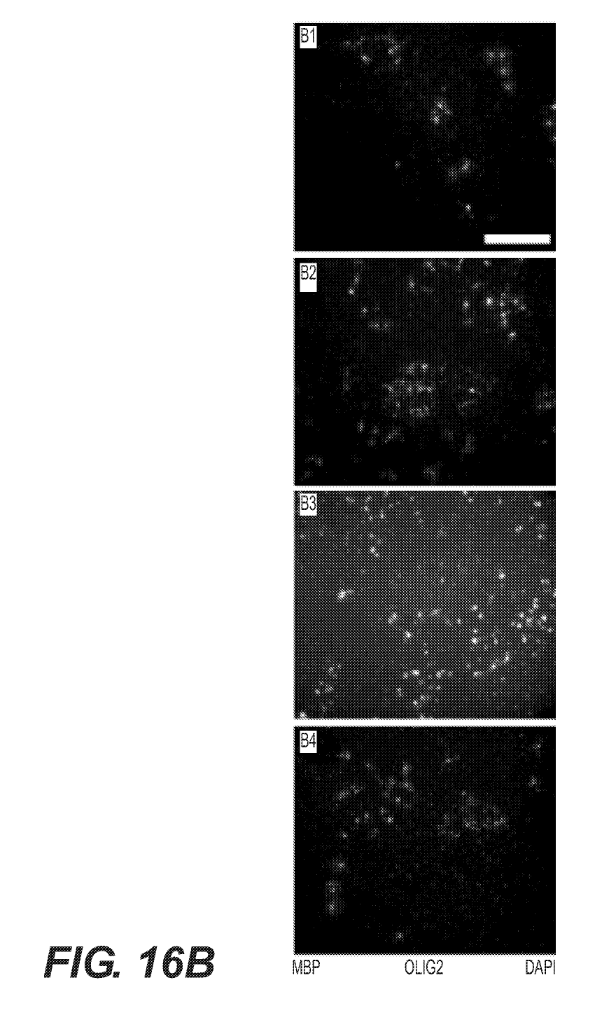

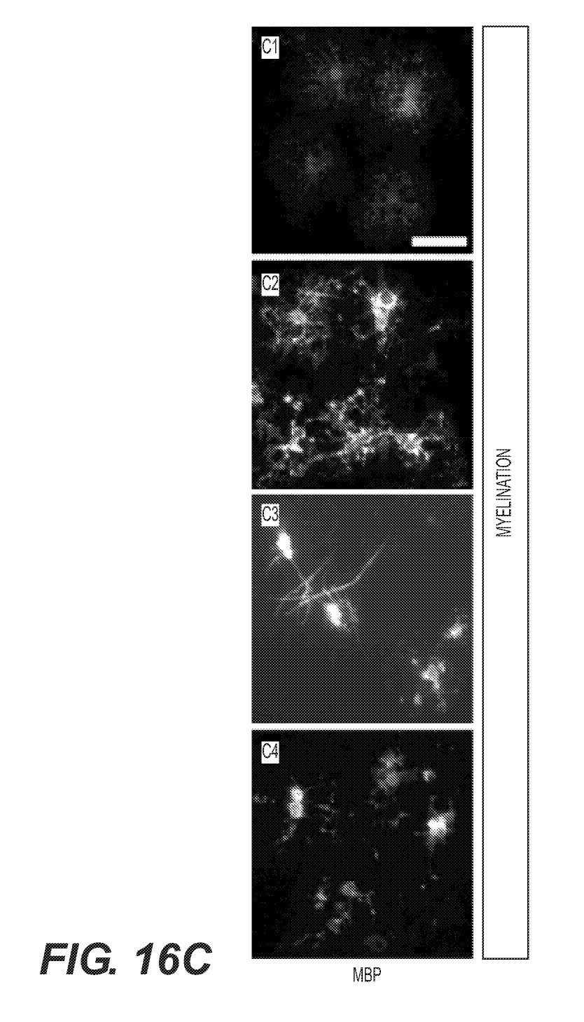

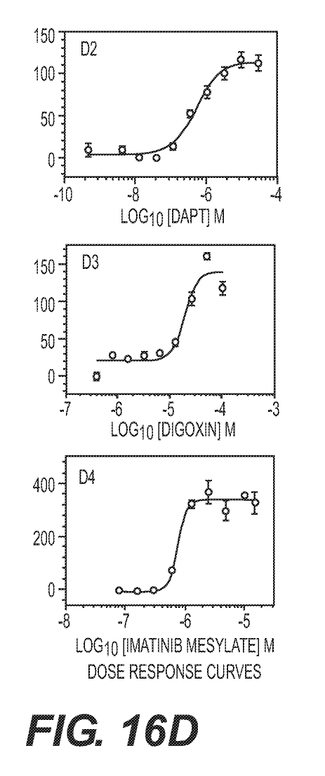

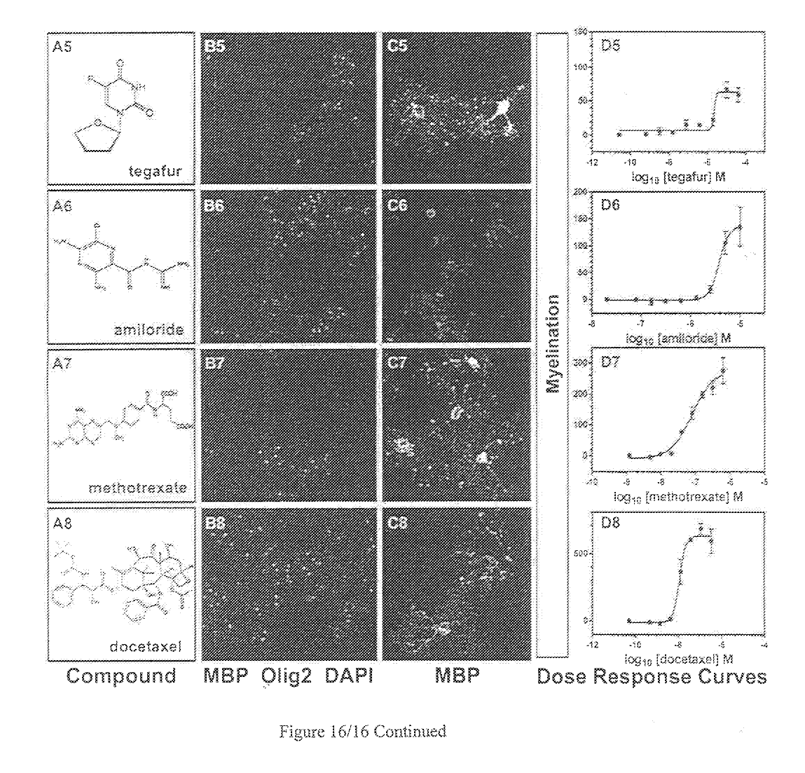

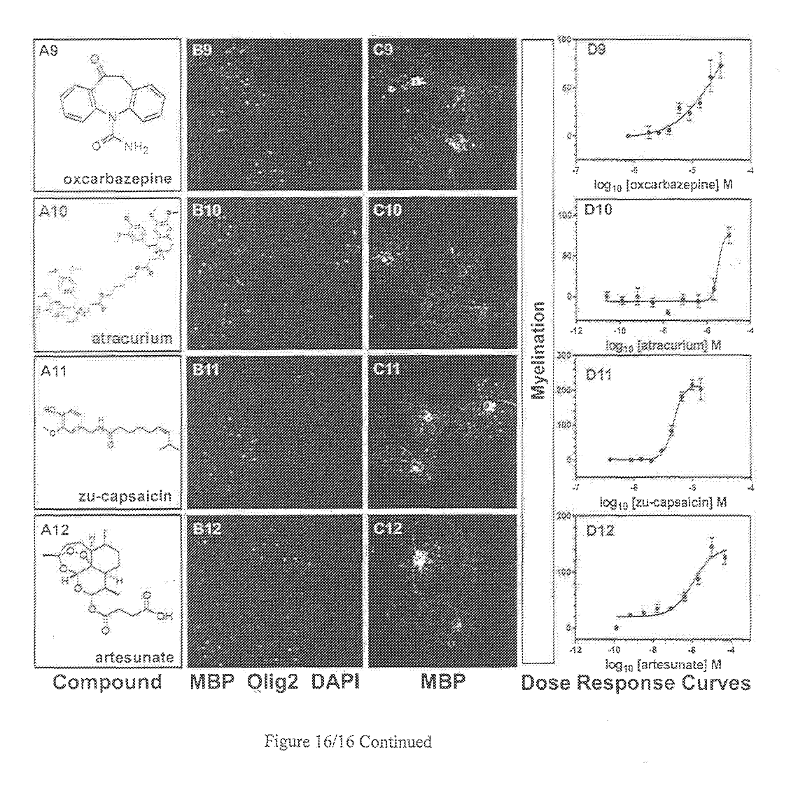

Following the differentiation and growth of neurons and glia for five days, the differentiation and early myelination of exogenously added OPCs was determined to proceeded optimally when the growth medium was switched from NB/N21 to an OPC-supporting myelination medium (MyM). With this growth medium, it was observed that it was not necessary to add exogenous OPCs as mature and axon ensheathing OLs that had differentiated from the embryonic cortical preparation, presumably from neural precursor cells and/or OPCs could be readily identified. It was actually found that the differentiating OPCs already present in the cultures produced better myelination (FIG. 7, see below and methods for quantification of myelination). Finally, it was determined that the optimal time course for myelination to proceed was eight days after test compound addition and 13 DIV total (FIG. 8). FIG. 1 depicts the flow scheme of the embryonic cortical cell assay. At this early myelination time point, we observed MBP staining aligning with SMI 31/32 axon staining, indicating that indeed OLs are contacting and aligning with axons (FIG. 2A).

The γ-secretase inhibitor, DAPT, a known enhancer of myelination [11], [9] was utilized as a positive control to test the assay system and establish an automated morphology analysis. After compound treatment, cells were stained for the OL lineage marker, Olig2, myelin basic protein (MBP) to stain mature OLs, and the nuclear dye, DAPI, and imaged. Myelination was scored by quantifying the characteristic change of morphology of OLs when ensheathing axons—from many branched, flattened, and diffusely MBP stained processes to condensed and aligned MBP-positive fibers. For each high resolution 10× image, we quantified the total length of contiguous, aligned MBP staining (fiber length)/number of Olig2-positive (Olig2⁺) nuclei, referred to as myelination). FIG. 2B demonstrates the digital mask created by the protocol used in the fiber length calculation. With these methods, significant dose-dependent increases in myelination with DAPT was determined(FIG. 2C). Importantly, reproducible EC₅₀ values of four GSI compounds, DAPT, LY411,575, BMS 708,163, and MRK560, were determined allowing the ranking of compounds (FIG. 3A, 3B, 3C, and 3D, Table 1). GSI-mediated facilitation of myelination was only observed in the presence of live axons and had no effect on the differentiation of purified OPCs grown in isolation (FIG. 9). Two other compounds identified from published high throughput library screens that promote OL differentiation in cultures containing purified OPCs, benztropine and clemastine [2],[3] were tested. As expected, these compounds demonstrated significant OL differentiation in the acutely prepared OL differentiation assay (FIG. 9). However, in the cortical myelination assay, benztropine and clemastine did not promote myelination (FIG. 10). This data demonstrates that the cortical myelination assay identifies novel compounds with myelination activity distinct from compounds that solely promote OL differentiation.

### Long-Term Characterization of Cortical Myelination Cultures

To test whether the enhanced early myelination effects of GSIs had longer lasting effects with the single eight day drug treatment course, cortical cultures were treated as described followed by two weeks of half medium changes with fresh MyM without GSI compound. These GSI-treated cultures demonstrated robust MBP alignment compared to DMSO vehicle controls (FIG. 4A and 4B). Additionally, these cortical cultures were tested for their ability to initiate the formation of axonal nodes of Ranvier, essential to action potential propagation in functionally myelinated axons. As an indicator of early node formation, these longer term cultures were immunostained with antibodies to the paranode-localized protein, Caspr [12] along with anti-MBP antibodies. FIG. 4C demonstrates the accumulation of Caspr protein at the edges of myelinated axon segments indicating that the initiation of node formation was induced by contact with OL myelin. These data demonstrate the ability of cortical cultures to form robustly myelinated axon segments and initiate node formation which is enhanced by an early, single dose treatment with GSIs.

### Cellular Composition of Cortical Myelination Cultures

To determine the composition of these cortical cultures, well-established cell type marker antibodies were used to identify different cell populations. Cultures were grown using the culture conditions described above, and fixed on DIV13. All cultures were stained with the nuclear marker DAPI to identify the total population of all the cells in culture. To identify the neuronal population, anti-NeuN antibodies were used to identify neuronal nuclei, as well as anti-MAP2 and anti-SMI 31/32 neurofilament antibodies to assess the health and extent of dendrite and axon formation, respectively. Imaging of neurons in these cultures demonstrated mature cortical neurons with well-developed dendrites and a dense bed of axons (FIG. 11). In addition to NeuN for the identification of neurons, anti-Olig2 antibodies were used to identify OPC/OLs and anti-GFAP antibodies to identify astrocytes. The percentage of each of these cell types in this cortical co-culture preparation was then quantified as a percentage of the total cell population identified with DAPI nuclear staining of all cells. It was found that the cell composition under these culture conditions was 23% neurons, 46% astrocytes, 22% OPCs/OLs, and 9% unidentified cells.

In order to better understand how OLs differentiate and develop in these cultures, DIVS cultures were stained and imaged to assess the cell composition of our cultures on the day of test compound addition. At this stage, the cultures contained ˜50% neurons, having already generated an axon network (FIG. 12). Since the cultures were derived from embryonic cortex, the bi-potent O2A glial progenitor antibody marker A2B5 [13], [14], was used to identify glial progenitors still capable of differentiating. DIVS cultures contained abundant A2B5 positive cells which were not observed at DIV13 (FIG. 13). There were relatively few astrocytes (positively staining for GFAP) and differentiated OLs (positively staining for MBP, CNP, O4 or MOG) at this stage, indicating that a majority of the OL differentiation and myelination occurred during the test compound treatment window (FIG. 14). Using the microglial marker lba1, we detected <1% microglial cells at DIVS, and undetectable microglia at DIV13.

Screening for Compounds that Promote Myelination

To demonstrate that the assay conditions developed were robust enough to support drug discovery screening efforts, a small library of compounds were screended. The NIH Clinical Collection (NCC) library was selected for screening which contains Food and Drug Administration (FDA)-approved off-patent drugs. Therefore, hits retrieved from this collection could lead to potential drug candidates for further development and rapid repositioning as therapeutics for MS. The NCC library consists of 727 biologically active compounds that have been through phase I-III clinical trials. This collection is additionally attractive because of the wide variety of cellular targets that are represented. Because this focused FDA-approved compound collection is small and the drug structures diverse, two concentrations (5 μM and 1 μM) were screened to reduce the possibility of missing hits due to false negatives. Each plate contained eight wells treated with DMSO or DAPT controls and each test compound concentration was screened in duplicate.

Automated image acquisition was performed from four randomized fields from each well, representing a total of eight data points per test concentration. The data was analyzed to find active compounds that increase myelin formation above a pre-defined threshold (>50% of DAPT pro-myelinating activity). The delineation of three SDs above the mean signal of DMSO-treated well was included (FIG. 5A). While not a criterion in the assay for hit selection, it provided a statistical assurance that we were well out of a false-positive hit rate (0.15%) range. Control DAPT versus DMSO values from the entire myelination screen were highly statistically significant (FIG. 5B) indicating an acceptable screen window.

Given the possibility of inter-preparation variability and differences in the behaviors of dissociated primary neurons and glia in culture, so we how to evaluate the consistency of responses to treatment in addition to assessing the consistency of the plate controls was considered. As a measure of assay quality, the Z-factor was considered; however, since the assay is based on multiple readouts, an additional parameter was incorporated for assay window and robustness, a specific morphological measurement of early myelination. This criterion is a quotient of two morphological measurements—fiber length intensity score and MBP intensity score which adjusts for the contribution of OL differentiation (MBP expression) (FIG. 15). A ratio of one indicates that an observed increase in myelination may almost be entirely accounted for by an increase in the extent of OL differentiation, whereas a value significantly greater than one indicates that there is an observed increase in myelination above and beyond what would be expected by an increase in OL differentiation alone, i.e. specific induction of myelination. This was primarily used to measure the quality of each plate in the screen. It was typically observed DAPT treated cultures have fiber/MBP scores >1.5. FIG. 5C depicts the DAPT/DMSO fiber/MBP scores of each plate from the entire library screen which generated an average fiber/MBP score of 1.61. For each plate in the screen, the acceptable fiber/MBP ratio cutoff was in the range of 1.3±0.2. In addition, the fiber/MBP score was incorporated into the criteria for assay hits to potentially distinguish between active compounds with distinct mechanisms of action (see below).

### 7.5 Selection Criteria For Myelination Assay Positive Hit Compounds

To select candidate compounds with substantial activity in our screen, compounds that had scores >50% of DAPT were included as secondary hits (FIG. 5D). In addition, to further delineate the myelination effect enhanced by compounds, we implemented a second criterion of fiber length/MBP (FIG. 15) including any compounds that had scores >1.5. Three compounds passed this criterion alone and were included in further hit compound refinement. We observed that some compounds displayed unusually large myelination scores but had very low overall Olig2+ nuclei expression, which reflects an inhibition of OL proliferation (e.g. methotrexate, FIG. 16B7). Not surprisingly, visual assessment of images from these cultures revealed a low overall number of myelinating OLs. We therefore incorporated a third criterion to eliminate compounds that primarily act as anti-proliferative compounds and thus greatly reduced Olig2 expression: the ratio of total nuclei (DAPI staining)/Olig2⁺ nuclei. Large DAPI/Olig2⁺ nuclei numbers (>40) were a clear indicator that the test compound severely depleted OPCs and OLs, undesirable in a screen for compounds that promote myelination. DAPT reduces the number of Olig2⁺ cells by ˜50%, most likely by promoting OPC differentiation and reducing OPC proliferation [9]. Therefore, we implemented a criterion of >25% of the DAPT Olig2⁺ cell count which also effectively eliminated compounds that severely reduced the number of Olig2⁺ cells (FIG. 5D). A fourth criterion was the qualitative assessment of OL MBP staining, taking into account the number of OLs/image field and OL morphology. Compounds that dramatically changed OL morphology (e.g. greatly enlarging the cell) while reducing the number of OLs/field were eliminated. Active compounds that passed all of these criteria were referred to as refined hits (FIG. 5D). A fifth criterion was to confirm activity and potency of refined hit compounds with full dose-response curve experiments of at least two replicates using reordered or resynthesized material. Actives that met this criterion were referred to as our confirmed hits (FIG. 5D) and our hit rate is based on this number. In a screen of 727 FDA-approved drugs, our screen identified 53 primary hits, 33 refined secondary hits and ten confirmed and reproducible hits (Table 1). The resulting hit rate for the entire screen was ˜1.7%. FIG. 16 shows the chemical structure of each hit compound, screening image of MBP/Olig2/DAPI staining, and the EC₅₀ curves for myelination. Table 1 shows the calculated myelination EC₅₀ values for the top hits from our cortical myelination screen. Based on the available literature on these previously characterized compounds, we grouped the hits based on the known mechanisms of action. These compounds fell into many different classes, grouped in Table 1, and are distinct from compounds previously identified by other library screens that have used OL differentiation assays in the absence of axons [2],[3],[4].

Methods Reagents: Dulbecco's modified Eagle Medium (DMEM) high glucose, Neurobasal medium (NB), Hank's balanced Salt Solution (HBSS), Earle's balanced Salt Solution, L-glutamine, sodium pyruvate, penicillin/streptomycin, Diamidino-2-Phenylindole, Dilactate (DAPI) were purchased from Life Technologies (Carlsbad, Calif., USA), N21-MAX medium supplement from R&D Systems (Minneapolis, Minn., USA), normal goat and fetal bovine serum, forskolin, triiodothyronine (T3), vitamin B12, hydrocortisone, biotin, boric acid, apotransferrin, putrescine, progesterone, sodium selenite, poly-D-lysine, recombinant human insulin, bovine serum albumin and DMSO were obtained from Sigma-Aldrich (St. Louis, Mo., USA). Trace elements B and trypsin 0.05%-EDTA were purchased from Mediatech, Inc. (Manassas, Va., USA). Human ceruloplasmin was purchased from EMD Millipore (Billerica, Mass., USA). Recombinant human BDNF and CNTF were purchased from PeproTech (Rock Hill, N.J., USA). Laminin was obtained from Trevigen (Gaithersburg, Md., USA). DNase and papain were purchased from Worthington Biochemical Corporation (Lakewood, N.J., USA). Packard Viewplates 96-well were purchased from Perkin Elmer (Waltham, Mass., USA).

Cell Culture Methods: All animal work was carried out in strict accordance with the recommendations in the Guide for the Care and Use of Laboratory Animals of the National Institutes of Health. All animal protocols were approved by Institutional Animal Care and Use Committee (IACUC) at the Molecular Medicine Research Institute. Animals were either euthanized by CO₂ asphyxiation or decapitation.

RGC-OPC Culture Methods: RGCs were prepared from P6-P7 Sprague-Dawley rat pups (Charles River, Wilmington, Mass., USA), following the RGC immunopanning purification protocol as described in Watkins et al., 2008 [9]. On DIV11 of RGC culture, cortical OPCs were purified from P7 Sprague-Dawley rat pups, following the OPC immunopanning purification protocol (as described in [30]. Six days following test compound addition (17 DIV), cells were fixed, immunostained and imaged as described below.

Embryonic Cortical Culture Methods: The dissection of E18 rat (Charles River, Wilmington, Mass., USA) cortex is similar to that described previously [31], [32], [33] with some modifications. Briefly whole cortices from three embryos were collected in a petri dish containing HBSS. After carefully removing the meninges, the tissue was divided into cortical hemispheres, dissected and the non-cortical structures were removed. Cortical tissue was then digested in 7 U/ml papain dissolved in HBSS with 500 U/ml DNase I, and incubated for 30 minutes at 35° C. The enzymatic reaction was terminated with DMEM containing 10% FBS. The tissue was allowed to settle, supernatant was removed and tissue was triturated with a flame-polished glass Pasteur pipette in DMEM/10% FBS, 250 U/ml DNAse I until the tissue was completely dispersed. The dissociated cell suspension was centrifuged at 200×g for 5 minutes and supernatant replaced with plating medium (NB medium with 1× N21 supplement and 2 mM L-glutamine and 1% penicillin-streptomycin). Viable cells were counted using trypan blue exclusion and typically exceeded 80%. Isolated cells were seeded onto 96-well plates pre-coated with poly-D-lysine (10 μg/m1) and laminin (2 ␣/ml) at a density of 20,000 cells/well (2×10⁵ cell/cm³). Neurons were allowed to adhere, recover, mature and extend axons for three days. On the fourth day, the plating medium was diluted with an equivolume of myelination medium (MyM), as described in Watkins et al., 2008 [9] with minor modifications (see results). The following day, two-thirds of the medium was replaced with fresh MyM and test compound. The day after establishing the primary culture was defined as day 1 in vitro (DIV1).

Acute Oligodendrocyte Differentiation Assay: OPCs from P7 Sprague-Dawley rat pups were purified by immunopanning and cultured as described [30]. OPCs were plated at 5000 cells/well into pDL-Laminin coated 96-well TC plate wells and centrifuged at 200×g to facilitate cell attachment, survival, and assure even distribution of OPCs. Plated OPCs were pre-incubated for 1-2 hours at 37° C. in 10% CO₂ incubator, followed by addition of test compounds in quadruplicate. Controls were added in eight replicate wells, negative control=0.1% DMSO final concentration; positive control=40 ng/ml T3. The day of OPC plating was considered DIV0. On DIV4, cells were fixed, immunostained, and imaged as described below. Minor modifications include blocking cells with 10% normal goat serum (NGS)/0.4% Triton X-100 and staining overnight at 4° C. with rat anti-MBP antibodies diluted in 10% NGS/0.08% Triton X-100. OL differentiation was quantified by IN Cell Developer Toolbox image analysis software which calculated the MBP staining intensity of two images per well. The extent of OL differentiation was defined by the total threshold-selected area of MBP staining×MBP fluorescence intensity in this area divided by the total number of OLs (identified by DAPI nuclear staining).

Immunofluorescence Staining and Imaging: At the experimental end point, medium was removed leaving 50 μl/well using an ELX405 microplate washer (BioTek, Winooski, Vt., USA). Cells were then fixed for 14 min with paraformaldehyde solution to a final concentration of 4%. Following fixation, plates were washed with 1 ml PBS leaving 50 μl/well using the microplate washer. Cells were then blocked in blocking buffer (10% normal goat serum, 0.1% Triton X-100, antibody buffer (150 mM NaCl, 50 mM Tris Base, 1% BSA, 100 mM L-lysine, 0.004% sodium azide, pH 7.4), and stained with mouse anti-rat MBP antibody and anti-rabbit Olig2 diluted in blocking buffer overnight at 4° C. The cells were washed and incubated with secondary antibodies, and DAPI, 0.3 μM for 1 h at room temperature. After a final wash, 100 μl of PBS was added to each well and plates imaged. Images were captured with a Nikon Eclipse TE-2000-U microscope, Zyla cMOS megapixel camera (ANDOR Technology, Belfast, UK), fitted with an automated stage controlled by NIS Elements AR software 4.0 (Melville, N.Y., USA). An air 10× lens was used to capture four images per well with 16 bit resolution, 2560×2160 pixels. Images for each assay run were captured using identical camera settings. Images were exported as TIFF files for analysis and quantification.

Image Quantification: TIFF files were analyzed using a custom algorithm created with IN Cell Investigator Developer Toolbox (GE Health Sciences, Piscataway, N.J., USA). For each well, four images were analyzed and the data from the duplicate well combined and averaged (total of eight images per test condition). The extent of OL differentiation was defined by the total threshold-selected area of MBP staining×MBP fluorescence intensity in this area divided by the total number of OLs (identified by Olig2 nuclear staining). We referred to this as the “MBP score” or “OL differentiation”. Earlier publications have characterized in vitro myelination as contiguous segments of MBP staining co-localizing with axons, representing the contact and ensheathment of axons with the myelin membrane generated by OLs [9]. Hence, our assay defined myelination as the alignment of MBP staining into contiguous segments, and the length of those contiguous segments was quantified. This was performed by first defining and selecting the area of MBP fluorescence intensity, followed by morphometric analysis of these areas using the “fiber length” algorithm (calculates the total pixel length within a single fibrous shape). This value was then divided by the total number of OLs to give the value referred to as “fiber score” or “myelination”. The quotient of the myelination score and the MBP score equals a value we referred to as “fiber/MBP ratio”, reflecting myelination independent of the effects of differentiation. Numerical results from the analyzed images were later exported for analysis in Microsoft Excel (Redmond, Wash., USA). Data was normalized by fitting parameters to positive (1 μM DAPT) and negative controls (0.1% DMSO) and expressed as the % of DAPT.

Relative EC₅₀ Analysis: Half maximal effect concentrations (EC₅₀) values were obtained by fitting the data to a sigmoidal dose-response curve-fitting function (Prism, GraphPad, San Diego, Calif., USA). Serial dilutions of eight to ten different concentrations with four data points per concentration were used for curve fitting. Experiments were repeated at least two times.

Compounds: All compounds in the NCC library were supplied in DMSO at 10 mM in 96-well plates. Hit compounds were purchased as powders and stocks dissolved in DMSO to 10 mM for in vitro studies. N-[N-(3,5-Difluorophenacetyl)-L-alanyl]-S-phenylglycine t-butyl ester (DAPT), LY411,575, and BMS 708163 were from Selleckchem, MRK560 was purchased from Tocris.

Statistical Methods: For all experiments, assuming normal distribution, two-tailed t-tests were used to evaluate comparisons between two groups and ANOVA was used when more than two groups were compared. For the quantitative analysis of in vitro myelination and differentiation, ANOVA with Bonferroni or Dunnett correction was used. Where possible, data were represented as mean±standard error of the mean (SEM) or standard deviation (SD) unless otherwise indicated in the figure legends.

Other embodiments and uses of the invention will be apparent to those skilled in the art from consideration of the specification and practice of the invention disclosed herein. All references cited herein, including all publications, and all U.S. and foreign patents and patent applications are specifically and entirely incorporated by reference. The term comprising, where ever used, is intended to include the terms consisting and consisting essentially of. Furthermore, the terms comprising, including, and containing are not intended to be limiting. It is intended that the specification and examples be considered exemplary only with the true scope and spirit of the invention indicated by the claims.

### REFERENCES

1. National Institute of Neurological Disorders and Stroke, Multiple Sclerosis: Hope Through Research [http://www.ninds.nih.gov/disorders/multiple_sclerosis/detail_multiple_scierosis.htm]
2. Mei F, Fancy S P, Shen Y A, Niu J, Zhao C, Presley B, Miao E, Lee S, Mayoral S R, Redmond S A et al: Micropillar arrays as a high-throughput screening platform for therapeutics in multiple sclerosis. Nat Med 2014, 20(8):954-960.
3. Deshmukh V A, Tardif V, Lyssiotis C A, Green C C, Kerman B, Kim H J, Padmanabhan K, Swoboda J G, Ahmad I, Kondo T et al: A regenerative approach to the treatment of multiple sclerosis. Nature 2013, 502(7471):327-332.
4. Najm F J, Madhavan M, Zaremba A, Shick E, Karl R T, Factor D C, Miller T E, Nevin Z S, Kantor C, Sargent A et al: Drug-based modulation of endogenous stem cells promotes functional remyelination in vivo. Nature 2015.
5. Demerens C, Stankoff B, Logak M, Anglade P, Allinquant B, Couraud F, Zalc B, Lubetzki C: Induction of myelination in the central nervous system by electrical activity. Proc Natl Acad Sci U S A 1996, 93(18):9887-9892.
6. Lubetzki C, Demerens C, Anglade P, Villarroya H, Frankfurter A, Lee V M, Zalc B: Even in culture, oligodendrocytes myelinate solely axons. Proc Natl Acad Sci U S A 1993, 90(14):6820-6824.
7. De Strooper B, Annaert W, Cupers P, Saftig P, Craessaerts K, Mumm J S, Schroeter E H, Schrijvers V, Wolfe M S, Ray W J et al: A presenilin-1-dependent gamma-secretase-like protease mediates release of Notch intracellular domain. Nature 1999, 398(6727):518-522.
8. Dovey H F, John V, Anderson J P, Chen L Z, de Saint Andrieu P, Fang L Y, Freedman S B, Folmer B, Goldbach E, Holsztynska E J et al: Functional gamma-secretase inhibitors reduce beta-amyloid peptide levels in brain. J Neurochem 2001, 76(1):173-181.
9. Watkins T A, Emery B, Mulinyawe S, Barres B A: Distinct stages of myelination regulated by gamma-secretase and astrocytes in a rapidly myelinating CNS coculture system. Neuron 2008, 60(4):555-569.
10. Lee S, Leach M K, Redmond S A, Chong S Y, Mellon S H, Tuck S J, Feng Z Q, Corey J M, Chan J R: A culture system to study oligodendrocyte myelination processes using engineered nanofibers. Nat Methods 2012, 9(9):917-922.
11. Jurynczyk M, Jurewicz A, Bielecki B, Raine C S, Selmaj K: Inhibition of Notch signaling enhances tissue repair in an animal model of multiple sclerosis. J Neuroimmunol 2005, 170(1-2):3-10.
12. Buttermore E D, Thaxton C L, Bhat M A: Organization and maintenance of molecular domains in myelinated axons. J Neurosci Res 2013, 91(5):603-622.
13. Eisenbarth G S, Walsh F S, Nirenberg M: Monoclonal antibody to a plasma membrane antigen of neurons. Proc Natl Acad Sci U S A 1979, 76(10):4913-4917.
14. Hunter S F, Bottenstein J E: Bipotential glial progenitors are targets of neuronal cell line-derived growth factors. Brain Res Dev Brain Res 1989, 49(1):33-49.
15. Najm F J, Zaremba A, Caprariello A V, Nayak S, Freundt E C, Scacheri P C, Miller R H, Tesar P J: Rapid and robust generation of functional oligodendrocyte progenitor cells from epiblast stem cells. Nat Methods 2011, 8(11):957-962.
16. Lee X, Yang Z, Shao Z, Rosenberg S S, Levesque M, Pepinsky R B, Qiu M, Miller R H, Chan J R, Mi S: NGF regulates the expression of axonal LINGO-1 to inhibit oligodendrocyte differentiation and myelination. J Neurosci 2007, 27(1):220-225.
17. Ishibashi T, Dakin K A, Stevens B, Lee P R, Kozlov S V, Stewart C L, Fields R D: Astrocytes promote myelination in response to electrical impulses. Neuron 2006, 49(6):823-832.
18. Jepson S, Vought B, Gross C H, Gan L, Austen D, Frantz J D, Zwahlen J, Lowe D, Markland W, Krauss R: LINGO-1, a transmembrane signaling protein, inhibits oligodendrocyte differentiation and myelination through intercellular self-interactions. J Biol Chem 2012, 287(26):22184-22195.
19. Fischer R, Wajant H, Kontermann R, Pfizenmaier K, Maier O: Astrocyte-specific activation of TNFR2 promotes oligodendrocyte maturation by secretion of leukemia inhibitory factor. Glia 2014, 62(2):272-283.
20. Bonora M, De Marchi E, Patergnani S, Suski J M, Celsi F, Bononi A, Giorgi C, Marchi S, Rimessi A, Duszynski J et al: Tumor necrosis factor-alpha impairs oligodendroglial differentiation through a mitochondria-dependent process. Cell Death Differ 2014, 21(8):1198-1208.
21. Williams S K, Maier O, Fischer R, Fairless R, Hochmeister S, Stojic A, Pick L, Haar D, Musiol S, Starch M K et al: Antibody-mediated inhibition of TNFR1 attenuates disease in a mouse model of multiple sclerosis. PLoS One 2014, 9(2):e90117.
22. Barres B A, Lazar M A, Raff M C: A novel role for thyroid hormone, glucocorticoids and retinoic acid in timing oligodendrocyte development. Development 1994, 120(5):1097-1108.
23. Kaji R, Happel L, Sumner A J: Effect of digitalis on clinical symptoms and conduction variables in patients with multiple sclerosis. Ann Neurol 1990, 28(4):582-584.
24. Azizi G, Haidari M R, Khorramizadeh M, Naddafi F, Sadria R, Javanbakht M H, Sedaghat R, Tofighi Zavareh F, Mirshafiey A: Effects of imatinib mesylate in mouse models of multiple sclerosis and in vitro determinants. Iran J Allergy Asthma Immunol 2014, 13(3):198-206.
25. Wang Z, Qiu J, Guo T B, Liu A, Wang Y, Li Y, Zhang J Z: Anti-inflammatory properties and regulatory mechanism of a novel derivative of artemisinin in experimental autoimmune encephalomyelitis. J Immunol 2007, 179(9):5958-5965.
26. Mueller A M, Nassery A, Conlon H, Liu X, Jun E, Yoon B H, Cristofanilli M, Sadiq S A: Effects of intraventricular methotrexate administration on Cuprizone-induced demyelination in mice. Front Mol Neurosci 2013, 6:34.
27. Ashtari F, Savoj M R: Effects of low dose methotrexate on relapsing-remitting multiple sclerosis in comparison to Interferon beta-1alpha: A randomized controlled trial. J Res Med Sci 2011, 16(4):457-462.
28. Al-Izki S, Pryce G, Hankey D J, Lidster K, von Kutzleben S M, Browne L, Clutterbuck L, Posada C, Edith Chan A W, Amor S et al: Lesional-targeting of neuroprotection to the inflammatory penumbra in experimental multiple sclerosis. Brain 2014, 137(Pt 1):92-108.
29. Acar A, Nuri Deniz M, Erhan E, Ugur G: Anesthetic technique in a patient with multiple sclerosis scheduled for laparoscopic nephrectomy for a renal tumor: a case report. Anesth Pain Med 2013, 2(3):138-140.
30. Dugas J C, Tai Y C, Speed T P, Ngai J, Barres B A: Functional genomic analysis of oligodendrocyte differentiation. J Neurosci 2006, 26(43):10967-10983.
31. Noh J S, Gwag B J: Attenuation of oxidative neuronal necrosis by a dopamine D1 agonist in mouse cortical cell cultures. Exp Neurol 1997, 146(2):604-608.
32. Ratan R R, Ryu H, Lee J, Mwidau A, Neve R L: In vitro model of oxidative stress in cortical neurons. Methods Enzymol 2002, 352:183-190.
33. Kraus R L, Pasieczny R, Lariosa-Willingham K, Turner M S, Jiang A, Trauger J W: Antioxidant properties of minocycline: neuroprotection in an oxidative stress assay and direct radical-scavenging activity. J Neurochem 2005, 94(3):819-827.
34. Barbara Costa, Gabriella Giagnoni, Chiara Franke, Anna Elisa Trovato and Mariapia Colleoni: Vanilloid TRPV1 receptor mediates the antihyperalgesic effect of the nonpsychoactive cannabinoid, cannabidiol, in a rat model of acute inflammation. Br J Pharmacol. 2004, 143(2): 247-250.
35. Lannotti F A, Hill C L, Leo A, Alhusaini A, Soubrane C, Mazzarella E, Russo E, Whalley B J, Di Marzo V, Stephens G J: Nonpsychotropic plant cannabinoids, cannabidivarin (CBDV) and cannabidiol (CBD), activate and desensitize transient receptor potential vanilloid 1 (TRPV1) channels in vitro: potential for the treatment of neuronal hyperexcitability. ACS Chem Neurosci. 2014, 5(11):1131-41.

All cited references are incorporated herein by reference in their entirety.

TABLE 1
Confirmed Hits from the NCC Library Screen

| Known mechanism of | | |
|---|---|---|
| action | Compound | EC50 mM avg † |
| Kinase inhibitor | Imatinib mesylate | 1.4 |
| Anti-cholinergic | Atracurium Besylate | 5.3 |
| Mitotic inhibitors | Docetaxel | 0.1 |
| | Methotrexate | 0.1 |
| | Tegafur | 2.7 |
| | Artesunate | 3.3 |
| Ion channels | Zu-capsaicin | 4.7 |
| | Amiloride | 8.9 |
| | Oxcarbazepine | 14.7 |
| Na²⁺/K+ ATPase inhibitor | Digoxin | 11.3 |
| γ-secretase inhibitors | LY 411, 575* | 0.00053 |
| | BMS 708, 163* | 0.067 |
| | MRK 560* | 0.082 |
| | DAPT* | 0.55 |

*Compounds not part of the NCC library screen.
† N = ≥2

1.-19. (canceled)

20. A method comprising:
providing one or more TRPV1 agonists exhibiting promyelinating activity; and
administering an effective amount of the one or more TRPV1 agonists to a subject.

21. The method of claim 20, wherein administering results in repairing and/or maintaining the myelin sheath of neuronal axons of the subject.

22. The method of claim 20, wherein administering results in promoting myelination of an axon of a nerve cell.

23. The method of claim 20, wherein administering results in treating a demyelinating disorder of a subject.

24. The method of claim 20, wherein administering results in neuroprotection.

25. The method of claim 20, wherein the one or more TRPV1 agonists exhibiting promyelinating activity are selected from the group consisting of zu-capsaicin, capsaicin, cannabinoids, such as cannabidivarin and cannabidiol, anadamide, vanilloids and combinations thereof.

26. The method of claim 25, wherein the one or more TRPV1 agonists exhibiting promyelinating activity comprise zu-capsaicin.

27. The method of claim 20, further comprising administration of one or more additional therapeutics.

28. The method of claim 27, wherein the one or more additional therapeutics are selected from the group consisting of anti-inflammatory agents, immune modulators, additional agents having promyelinating activity, and combinations thereof.

29. The method of claim 28, wherein the subject has a central nervous system (CNS) demyelinating disorder.

30. The method of claim 29, wherein said subject has multiple sclerosis.

31. The method of claim 1, wherein the one or more TRPV1 agonists exhibiting promyelinating activity are formulated for intranasal administration.

32. A composition comprising one or more TRPV1 agonists exhibiting promyelinating activity.

33. The composition of claim 32, which promotes myelination of an axon of a nerve cell comprising.

34. The composition of claim 32, which effectively treats a demyelinating disorder.

35. The composition of claim 32, which is effective for neuroprotection.

36. The composition of claim 32, wherein the one or more TRPV1 agonists are selected from the group consisting of zu-capsaicin, capsaicin, cannabinoids, such as cannabidivarin and cannabidiol, anadamide, vanilloids and combinations thereof.

37. The composition of claim 36, wherein the one or more TRPV1 agonists comprise zu-capsaicin.

38. The composition of claim 32, further comprising one or more additional therapeutics.

39. The composition of claim 38, wherein the one or more additional therapeutics are selected from the group consisting of anti-inflammatory agents, immune modulators, additional agents having promyelinating activity, and combinations thereof.
